SEQUENCE LISTING (5 sequences)

SEQ ID NO: 1 - DNA, 307 bp, Artificial Sequence
  307 bp optimised NPC1 promoter sequence
cgagccagactccataagtcccgcgcctggcccccggggattgcaggggctgaggagaagggcaacacggggaccttgaagcggggtcgcggcggcgccccagcccgggccagggagtcccggcagcggcacctcccagaaagggcggagccgacgacgccttcttccttcctgaccggcgcgcgcagcctgctgccgcggtcagcgcctgctcctgctcctccgctcctcctgcgcggggtgctgaaacagcccggggaagtagagccgcctccggggagcccaaccagccgaacgccgccggcgt

SEQ ID NO: 2 - DNA, 4760 bp, Homo sapiens
cttcctgaccggcgcgcgcagcctgctgccgcggtcagcgcctgctcctgctcctccgctcctcctgcgcggggtgctgaaacagcccggggaagtagagccgcctccggggagcccaaccagccgaacgccgccggcgtcagcagccttgcgcggccacagcatgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatgaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctagccctctcgcagggcatcctgactgaactgtgtctaagggtcggtcggtttaccactggacgggtgctgcatcggcaaggccaagttgaacaccggatggtgccaaccatcggttgtttggcagcagctttgaacgtagcgcctgtgaactcaggaatgcacagttgacttgggaagcagtattactagatctggaggcaaccacaggacactaaacttctcccagcctcttcaggaaagaaacctcattctttggcaagcaggaggtgacactagatggctgtgaatgtgatccgctcactgacactctgtaaaggccaatcaatgcactgtctgtctctccttttaggagtaagccatcccacaagttctataccatatttttagtgacagttgaggttgtagatacactttataacattttatagtttaaagagctttattaatgcaataaattaactttgtacacatttttatataaaaaaacagcaagtgatttcagaatgttgtaggcctcattagagcttggtctccaaaaatctgtttgaaaaaagcaacatgttcttcacagtgttcccctagaaaggaagagatttaattgccagttagatgtggcatgaaatgagggacaaagaaagcatctcgtaggtgtgtctactgggttttaacttatttttctttaataaaatacattgttttcctaagttttggggttaccctatctgctttgagagacaaatacaaaagctaaatggaagaga

SEQ ID NO: 3 - protein, 1278 aa, Homo sapiens
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHMKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF

SEQ ID NO: 4 - DNA, 4673 bp, Homo sapiens
tttgctcctgctcctccgctcctcctgcgcggggtgctgaaacagcccggggaagtagagccgcctccggggagcccaaccagccgaacgccgccggcgtcagcagccttgcgcggccacagcatgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatcaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctagccctctcgcagggcatcctgactgaactgtgtctaagggtcggtcggtttaccactggacgggtgctgcatcggcaaggccaagttgaacaccggatggtgccaaccatcggttgtttggcagcagctttgaacgtagcgcctgtgaactcaggaatgcacagttgacttgggaagcagtattactagatctggaggcaaccacaggacactaaacttctcccagcctcttcaggaaagaaacctcattctttggcaagcaggaggtgacactagatggctgtgaatgtgatccgctcactgacactctgtaaaggccaatcaatgcactgtctgtcctctcctttttaggagtaagccatcccacaagttctataccatatttttagtgacagttgaggttgtagatacactttataacattttatagtttaaagagctttattaatgcaataaattaactttgtacacatttttatataaaaaaacagcaagtgatttcagaatgttgtaggcctcattagagcttggtctccaaaaatctgtttgaaaaaagcaacatgttcttcacagtgttcccctagaaaggaagagatttaattgccagttagatgtggcatgaaatgagggacaaagaaagcatctcgtaggtgtgtctactgggttttaacttatttttctttaataaaatacattgttttcctaaaaaaaaaa

SEQ ID NO: 5 - protein, 1278 aa, Homo sapiens
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHIKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF

(19) United States
(12) Patent Application Publication
(10) Pub. No.: US 2023/0190960 A1
(43) Pub. Date: Jun. 22, 2023

(54) GENE THERAPY OF NIEMANN-PICK DISEASE TYPE C

(71) Applicant: UCL BUSINESS LTD, London (GB)

(72) Inventors: Michael HUGHES, London (GB); Ahad RAHIM, London (GB)

(21) Appl. No.: 17/910,686

(22) PCT Filed: Mar. 10, 2021
(86) PCT No.: PCT/GB2021/050597

§ 371 Date: Sep. 9, 2022

(30) Foreign Application Priority Data
Mar. 11, 2020 (GB) 2003536.6

(51) Int. Cl. A61K 48/00; A61P 3/00; A61K 38/17; C12N 15/86

(52) U.S. Cl. CPC A61K 48/0058; A61K 48/0008; A61P 3/00; A61K 38/1709; C12N 15/86; C12N 2750/14122; C12N 2750/14143; C12N 2750/14152; C12N 2750/14171

(57) ABSTRACT

The present invention relates to expression constructs and vectors for the treatment and/or prevention of diseases that are associated with a loss of NPC1 function, such as the lysosomal storage disorder Niemann-Pick type C (NPC) disease.

### FIELD OF THE INVENTION

The present invention relates to expression constructs and vectors for the treatment and/or prevention of diseases that are associated with a loss of NPC1 function, such as the lysosomal storage disorder Niemann-Pick type C (NPC) disease.

### BACKGROUND OF THE INVENTION

Niemann-Pick type C (NP-C) disease is a rare, fatal, autosomal recessive lysosomal storage disorder with neurological and visceral pathology, for which there is currently no major curative treatment (Vanier MT et al., 2010). NP-C is characterized by a progressive neurological degeneration that causes disability and premature death. Ninety five percent of cases are caused by a loss-of-function mutation to NPC1 (Vanier MT et al., 1996), which encodes the late endosomal transmembrane protein NPC1 (Higgins ME et al., 1999). NPC1 plays a role in intracellular lipid trafficking, with loss of NPC1 function leading to the accumulation of glycosphingolipids and cholesterol in endosomal compartments (Te Vruchte D et al., 2004), however premature death is usually associated with the neurological manifestations. There is no cure for NP-C but there is a disease-modifying drug (miglustat, Zavesca©), that partially inhibits glucosylceramide synthase, that slows disease progression but with associated side effects, including osmotic diarrhoea particularly during the first few weeks of administration.

More recently, Orphan Drug designation was granted to Arimoclomol© (Orphazyme AsP) as a potential treatment for Niemann-Pick type C patients. Arimoclomol© is a co-inducer of the heat-shock response that induces the expression of molecular chaperones like Hsp70, and activates natural cellular repair pathways. The treatment has already shown beneficial effects in pre-clinical studies on animal models of amyotrophic lateral sclerosis, spinal bulbar muscular atrophy and retinitis pigmentosa. The on-going phase 2 study (NCT02612129) is currently investigating the efficacy and safety of the drug on NP-C subjects. Since not all mutations will be responsive to potential chaperone therapy and the effects of the treatment may not always be sufficient, researchers are investigating the possibility of chaperone therapy in combination with other treatments.

Gene therapy using adeno-associated viral vectors (AAV) administered to the central nervous system as a potential treatment for neurodegenerative lysosomal storage disorders has transitioned from pre-clinical studies to clinical trials (NCT01801709, NCT00151216, NCT01414985, NCT02725580, NCT01474343 and ISRCTN19853672). For NP-C, there have been several published pre-clinical studies using AAV9 to partially ameliorate symptoms in the Npc1^{-/-} mouse models (Chandler R J et al., 2017, Xie C et al., 2017, Hughes M P et al (2018)).

There is, however, a need for improved therapies for Niemann-Pick type C (NPC) disease. The fact that many patients have disease onset in childhood makes the search for effective therapies urgent.

### SUMMARY OF THE INVENTION

The present invention is based on the creation of an optimised expression construct for expressing the NPC1 gene in cells.

In addition, the invention also relates to optimised gene therapy vectors for expressing the NPC1 gene in the brain and peripheral organs.

Constructs and vectors of the invention comprise a nucleic acid sequence encoding NPC1 and a NPC1 promoter fragment.

The large size of the NPC1 nucleic acid sequence makes packaging into an AAV vector along with a functional promoter problematic. Surprisingly, the inventors found that a NPC1 promoter fragment of less than 400 nucleotides in length was effective in expressing payload sequences such as GFP and NPC1 in the brain and peripheral organs. The NPC1 promoter fragment of the invention was especially effective in driving NPC1 expression to rescue function in Npc1^{-/-} mouse models. The inventors showed enhanced survival of NPC1 knock out mice following AAV9-hNPC1 treatment, with the NPC1 promoter extending the lifespan of the animals beyond all other tested promoters, including the CBA, CAG, and Synapsin (SYN) promoters.

Accordingly, the invention provides:

An expression construct comprising in a 5′ to 3′ direction:

(a) a NPC1 promoter fragment nucleotide sequence consisting of no more than 400 nucleotides in length, wherein the sequence comprises at least 250 consecutive nucleotides from SEQ ID NO: 1, or a sequence having at least 90% sequence identity to said promoter fragment sequence that retains the functionality of the NPC1 promoter; and

(b) (i) the hNPC1 nucleotide sequence as shown in SEQ ID NO: 2 or 4, or a sequence having at least 70% sequence identity to SEQ ID NO: 2 or 4 that retains the functionality of hNPC1; or (ii) a hNPC1 nucleotide sequence encoding the polypeptide as shown in SEQ ID NO: 3 or 5, or a polypeptide having at least 90% sequence identity to the polypeptide of SEQ ID NO: 3 or 5 that retains the functionality of hNPC1.

The invention also provides vectors and viral vectors comprising the expression constructs of the invention. The invention also provides host cells comprising the vectors or viral vectors of the invention. The invention also provides pharmaceutical compositions comprising the vectors of the invention and pharmaceutically acceptable carriers.

The invention also encompasses:

A vector of the invention or pharmaceutical composition of the invention for use in medicine.

A vector of the invention or pharmaceutical composition of the invention for use in a method of preventing or treating a disease associated with a loss of NPC1 function.

A vector of the invention or pharmaceutical composition of the invention for use in a method of preventing or treating lysosomal storage disorders such as Niemann-Pick disease type C (NPC) disease.

The invention also encompasses:

Use of a vector according to the invention or a pharmaceutical composition according to the invention in the manufacture of a medicament for the treatment or prevention of Niemann-Pick disease type C (NPC) disease.

The invention also encompasses:

A method of treating or preventing Niemann-Pick disease type C (NPC) disease in a patient in need thereof, comprising administering a therapeutically effective amount of a vector of the invention or a pharmaceutical composition of the invention to said patient; or

a method of treating or preventing lysosomal storage disorders such as Niemann-Pick disease type C (NPC) in a patient in need thereof, comprising administering a therapeutically effective amount of a vector of the invention or a pharmaceutical composition of the invention to said patient.

### BRIEF DESCRIPTION OF THE FIGURES

FIG. 1: In vitro promoter comparison with NLSeGFP reporter gene.

A). Transfection of plasmids containing selected promoters expressing nuclear localised eGFP in HEK293T cells. Due to the large size of hNPC1 cDNA and limited AAV packaging capacity, the optimal promoter should be <400 bp. Promoters longer than 500 bp (such as GAPDH, PGK and CAG) will likely cause packaging and truncation issues. B). Fluorescent images taken 72 hours post-transduction, capturing eGFP levels. Best eGFP expression by promoters under 400 bp was demonstrated by SYN-S (259 bp) and NPC-1 (307 bp). High activity from positive control CAG (643) also, which is too large. C). Quantification of the relative eGFP intensity from transduced cells (n=3 wells). Four small promoters (including NPC1 promoter) with activity comparable to original SYN promoter observed.

FIG. 2: Verification of neuronal and glial cell expression of NLSeGFP reporter gene in primary brain cultures in vitro.

Co-staining of primary brain cultures transduced with AAV9 vectors expressing nuclear localised eGFP reporter gene driven by the SYN, CAG or NPC1 promoter. Neuronal marker

(NeuN) and astrocyte marker (GFAP) indicate cell type and eGFP demonstrates reporter gene expression. White arrows indicate neuronal and glial cells that positively express the eGFP reporter gene.

Ubiquitous NPC1 promoter activity demonstrated in both neuronal and glial cells in vitro. No eGFP expression in astrocytes transduced with the neuronal AAV9. SYN.NLSeGFP vector, in comparison CAG and NPC promoters demonstrate positive expression in both cell types.

FIG. 3: In vivo promoter comparison with luciferase reporter gene.

A). Comparison of luciferase expression in brains and peripheral organs of P50 wildtype mice administered ICV neonataly with AAV9. NLSeGFP.2AFLuc under control of different promoters. B). Quantification of luciferase expression levels from promoters in analysed tissue. C). Heatmap of quantified luciferase expression levels in analysed tissue. NPC1 promoter activity demonstrated in vivo in both the brain at high levels and within peripheral organs. Synapsin short (SYNS) and NPC1 promoters show highest expression in the brain, with CAG expressing at lower levels. In general in peripheral organs CAG results in very high levels of reporter gene expression, NPC medium and SYNS the lowest.

FIG. 4: In vitro promoter comparison with human NPC1 cDNA.

A). Transfection of plasmids containing selected promoters expressing the human NPC1 cDNA in HEK293T cells. B). Western blot staining for NPC1 72 hours post-transfection, demonstrating NPC1 levels in cell lysates. Staining of B-Tubulin used as loading controls. C). Quantification of NPC1 band intensity for each promoter (n=3 blots). Confirmation of human NPC1 protein expression from NPC1 promoter confirmed in vitro. Highest levels of NPC1 expression from small promoters achieved with NPC1 promoter.

FIG. 5: Verification of neuronal and glial cell expression of NPC1 in primary brain cultures in vitro.

Co-staining of primary brain cultures transduced with AAV9 vectors expressing NPC1 riven by the SYN, CAL or NPC1 promoter. Neuronal marker (NeuN) and astrocyte marker (GFAP) indicate cell type and eGFP demonstrates NPC1 expression. White arrows indicate neuronal and glial cells that positively express NPC1. Positive NPC1 expression in both neuronal and glial cell types achieved with SYNS CAG and NPC1 promoters. Expression from SYN promoter limited to neurons. AAV9. CAG.FLUC used as negative control to indicate endogenous NPC1 levels. Confirmation of human NPC1 protein expression in neuronal and glial cells from NPC1 promoter.

FIG. 6: Evaluation of titre-matched AAV9-hNPC1 vectors with different promoters in Npc1^{-/-} knock out mouse model.

A). Normalisation of week 10 weights of NPC1 KO mice to wildtype levels achieved with SYN, NPC1, SYNS and CAG promoters. CBA promoter only partially improved weights compared to untreated KO mice. B). Enhanced survival of NPC1 KO mice following AAV9-hNPC1 treatment, with CBA promoter only showing partial rescue, SYN promoter showing doubling of lifespan and surprisingly the NPC promoter extends the lifespan beyond all other tested promoters. C). Western blot against NPC1 protein from half brain lysates of NPC1 KO mice treated with AAV9-hNPC1 vectors containing different promoters. SYN, SYNS, SYND and NPC promoters show high levels of NPC1 expression. Surprisingly the usually strong CAG promoter only achieves NPC1 expression just above wild type levels. NPC1 promoter demonstrates surprisingly high levels of NPC1 protein expression in the NPC1 KO mice compared to initial studies in in wildtype mice with reporter genes.

FIG. 7: Gait analysis of Npc1^{-/-} mice treated with titre-matched AAV9-hNPC1 vectors with different promoters.

A). Graphical representation of paw prints captured during an average run on CatWalk XT automated gait analyser by NPC1 KO mice at 10 weeks-of-age treated P0 ICV with AAV9-hNPC1 vector containing different promoters. B). Automated gait analysis quantification of 4 critical parameters affected in NPC1 KO mice. NPC1 KO mice treated with AAV9-hNPC1 containing the NPC1 promoter have normalised gait comparable to wildtype mice.

FIG. 8: Tremor analysis of Npc1^{-/-} mice treated with titre-matched AAV9-hNPC1 vectors with different promoters.

Quantification of high frequency tremor analysis of NPC1 KO mice at 10 weeks-of-age treated P0 ICV with AAV9-hNPC1 vector containing different promoters. NPC1 KO mice treated with AAV9-hNPC1 containing the NPC1 promoter have normalised tremor comparable to wildtype mice.

FIG. 9: In vivo promoter comparison of hNPC1 expression levels with P0 ICV AAV9-hNPC1 vectors.

A). Intracerebroventricular administration of AAV2/9 vectors (1.5E11 vg) into Npc1^{-/-} mice at P0/1, containing the human NPC1 cDNA under the expression of selected promoters.

These three constructs were chosen for initial in vivo evaluation as (1) CBA (273) is commonly used in clinical trials; (2) we have previously been using SYN (469) in preclinical studies; (3) NPC1 (307) showed promising activity at a small size. B). Anti-NPC1 immunohistochemistry on brain sections from age-matched wildtype control (Npc1^{+/+}), untreated knockout (Npc1^{-/}), and AAV9-hNPC1 treated knockout Npc1^{-/-} mice (P70). Following P0/1 ICV AAV9-hNPC1 delivery NPC1 (307) shows high levels of hNPC1 expression comparable to or higher than SYN (469) activity. NPC1 (307) promoter should additionally express in other neural cells, not limited solely to neurons as with SYN (469). Improvement in lifespan and behaviour demonstrated by both NPC1 (307) and SYN (469) vectors. No visible hNPC1 production from CBA (273) activity.

FIG. 10: Brain promoter comparison of hNPC1 expression levels with P0 ICV AAV9-hNPC1 vectors.

Comparison of hNPC1 protein expression via anti-NPC1 immunohistochemistry from AAV9-hNPC1 vectors containing different promoters within the brains of 10-week-old NPC1 KO mice injected P0 ICV.

Highest levels of NPC1 protein achieved with AAV9-hNPC1 vector containing the NPC1 promoter. Surprisingly, although NPC1 protein could be detected with the CAG containing AAV9-hNPC1 vector, the resulting NPC1 protein levels were low in comparison.

FIG. 11: Visceral organ promoter comparison of hNPC1 expression levels with PO ICV AAV9-hNPC1 vectors.

Comparison of hNPC1 protein expression via anti-NPC1 immunohistochemistry from AAV9-hNPC1 vectors containing different promoters within the visceral organs of 10-week-old NPC1 KO mice injected P0 ICV.

Although administration of the AAV9-hNPC1 vectors was via ICV injection, there is leakage of the vector into the periphery. Comparable to previous in vivo studies with reporter genes the CAG promoter showed the highest levels of visceral NPC1 protein expression. NPC1 expression from the NPC promoter was limited throughout visceral organs compared to the high levels observed in the brain.

FIG. 12: In vivo promoter comparison of Purkinje neuron survival with P0 ICV AAV9-hNPC1 vectors

Comparison of Purkinje neuron survival (their loss is hallmark of NPC disease pathology) via anti-Calbindin immunohistochemistry from AAV9-hNPC1 vectors containing different promoters within the cerebellum of 10-week-old NPC1 KO mice injected P0 ICV. AAV9-hNPC1 with NPC promoter induces the rescue of Purkinje neurons following treatment compared to untreated Npcl KO mice.

FIG. 13: In vivo evaluation of AAV9-NPC-NPC1 vector in point mutation NP-C model.

A). Comparison of hNPC1 protein expression via anti-NPC1 immunohistochemistry from AAV9-hNPC1 vector containing NPC1 promoter within the brains of 14-week-old Npcnmf164 mice injected P0 ICV. AAV9-NPC-hNPC1 induced positive expression of human NPC1 protein throughout the brain of Npc1nmf164 point mutation mice, which have a slower progression of NP-C disease compared to the KO model as they produce non-functional NPC1 protein (similar to the majority of patients). B). Evaluation of the effects of AAV9-hNPC1 vector containing the NPC1 promoter on Purkinje neuron loss (anti-Calbindin IHC), microglial activation (anti-CD68 IHC) and astrogliosis (anti-GFAP IHC) in the brains of Npc1nmf164 mice compared to untreated mice. AAV9-hNPC1 vector containing the NPC1 promoter results in rescue of Purkinje neurons along with reduction in neuroinflammation in the brain of treated Npc1nmf164 mice. Have demonstrated that this vector has therapeutic effects in 2 models of NP-C disease.

FIG. 14: In vivo evaluation of NPC1 protein expression from AAV9-NPC1 vector in point mutation NP-C model.

Analysis of hNPC1 expression via anti-NPC1 IHC in the brains and visceral organs of Npc1nmf164 mice treated with AAV9-hNPC1 containing either the SYN or NPC1 promoter, compared to untreated controls. AAV9-hNPC1 vector containing the short and ubiquitous NPC1 promoter results in extensive expression of NPC1 protein throughout the brains of treated NP-C mouse models, comparable to the larger neuron specific SYN promoter.

FIG. 15: In vivo evaluation of NPC1 promoter activity in non-neuronal neural cells.

Analysis of nuclear localised eGFP expression (green) in GFAP positive astrocytes (red) within the brains of wildtype mice injected P0 ICV with AAV9-NLSeGFP containing either the CAG or NPC1 promoter. AAV9-NLSeGFP vector containing the short and ubiquitous NPC1 promoter results in positive eGFP expression in GFAP positive astrocytes, comparable to the positive CAG control. No non-neuronal expression of GFP was observed with previous SYN promoter.

FIG. 16: In vivo comparison of NPC1 promoter activity in vivo in wildtype and NP-C mice.

A). Comparison of luciferase expression in brains and livers of 10-week-old mice wildtype and NPC1 KO mice administered ICV neonataly with AAV9. NLSeGFP.2AFLuc under control of the NPC promoter. B). Quantification of luciferase expression levels from analysed tissue.

Positive transgene expression achieved in both wildtype and NPC1 KO mice with NPC1 promoter. Interestingly, transgene expression on average was higher in the brains of NPC1 knockout mice compared to wildtype mice, indicating that NPC1 promoter activity may be higher in an environment of NP-C pathology.

FIG. 17: NPC1 promoter construct used in this study.

### BRIEF DESCRIPTION OF THE SEQUENCES

SEQ ID NO: 1—307 bp optimised NPC1 promoter sequence
SEQ ID NO: 2— human NPC1 nucleotide sequence NCBI Reference Sequence: NM_000271.5
SEQ ID NO: 3— human NPC1 protein sequence NCBI Reference Sequence: NP_000262.2
SEQ ID NO: 4— human NPC1 nucleotide sequence NCBI Reference Sequence AF002020.1
SEQ ID NO: 5— human NPC1 protein sequence NCBI Reference Sequence AAB63982.1

### DETAILED DESCRIPTION OF THE INVENTION

It is to be understood that different applications of the disclosed polynucleotide sequences may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural references unless the content clearly dictates otherwise. Thus, for example, reference to “a polynucleotide” includes “polynucleotides”, reference to “a promoter” includes “promoters”, reference to “a vector” includes two or more such vectors, and the like.

All publications, patents and patent applications cited herein, whether supra or infra, are hereby incorporated by reference in their entirety.

The present invention concerns gene therapy for the treatment and/or prevention of Niemann-Pick disease type C (NPC) disease.

The present invention also concerns gene therapy for the treatment and/or prevention of diseases that are associated with the loss or reduced function of NPC1. The present invention also concerns gene therapy for the treatment and/or prevention of diseases that are associated with the loss or reduced function of NPC1, including lysosomal storage disorders such as Niemann-Pick disease type C (NPC).

The present invention concerns gene therapy for the treatment and/or prevention of lysosomal storage disorders such as Niemann-Pick disease type C (NPC) in a patient in need thereof. The patient is preferably a mammal. The mammal may be a commercially farmed animal, such as a horse, a cow, a sheep or a pig, a laboratory animal, such as a mouse or a rat, or a pet, such as a cat, a dog, a rabbit or a guinea pig. The patient is more preferably human.

Lysosomal Storage Disorders

Lysosomal storage disorders are monogenic metabolic diseases caused by the accumulation of biological materials in the late endosome/lysosome system. These include more than 60 different diseases, and even though they are referred to as rare their estimated combined frequency at birth is 1:7,500.

Lysosomal storage disorders include Sphingolipidoses such as Fabry disease, Farber lipogranulomatosis, Gaucher disease type I, Gaucher disease types II and III, Niemann—Pick disease types A and B, GM1-gangliosidosis: infantile, juvenile and adult variants, GM2-gangliosidosis (Sandhoff): infantile and juvenile, GM2-gangliosidosis (Tay—Sachs): infantile, juvenile and adult variants, GM2-gangliosidosis (GM2-activator deficiency), GM3-gangliosidosis, Metachromatic leukodystrophy (late infantile, juvenile and adult) and Sphingolipid-activator deficiency; Mucopolysaccharidoses such as MPS I (Scheie, Hurler-Scheie and Hurler disease), MPS II (Hunter), MPS IIIA (Sanfilippo A), MPS TIM (Sanfilippo B), MPS IIIC (Sanfilippo C), MPS IIID (Sanfilippo D), MPS IVA (Morquio syndrome A), MPS IVB (Morquio syndrome B), MPS VI (Maroteaux—Lamy), MPS VII (Sly disease) and MPS IX; Glycogen storage diseases such as Pompe (glycogen storage disease type II); Oligosaccharidoses such as a-Mannosidosis, β-Mannosidosis, Fucosidosis, Aspartylglucosaminuria, Schindler disease, Sialidosis, Galactosialidosis, Mucolipidosis II (I-cell disease) and mucolipidosis III; Integral membrane protein disorders such as Cystinosis, Danon disease, Action myoclonus—renal failure syndrome, Salla disease, Niemann—Pick disease type C1 and Mucolipidosis IV; and disorders such as Multiple sulphatase deficiency, Niemann—Pick disease type C2, Wolman disease (infantile), cholesteryl ester storage disease and Galactosialidosis.

Traditionally, lysosomal storage diseases have been classified according to the substrate that accumulates in the cells. However, these diseases are mainly caused by mutations in the genes encoding enzymatic hydrolases involved in the metabolism of macromolecules, so that the same metabolic pathway can be affected in different pathologies. Therefore, although caused by different genetic defects, distinctive disorders could be characterised by the accumulation of the same biological material. Moreover, the identification of novel defects in lysosomal enzymes and integral proteins involved in trafficking broadened the traditional classification of lysosomal storage disorders.

The pathophysiology of lysosomal storage disorders is complex. The endosome/lysosome system is a tightly connected cellular compartment and it is responsible for the degradation and recycling of extracellular substrates. Moreover, cellular components, such as protein aggregates, damaged cytosolic organelles and intracellular pathogens can be targeted for degradation in lysosomes through the formation of autophagosomes and consequent fusion and release of the damaged cellular material into the lysosomal compartment. Autophagy is a tightly controlled cellular mechanism; therefore it is not surprising that this process is dysregulated in many lysosomal storage disorders. Indications of the involvement of impaired autophagy in lysosomal storage disorders have been found in several animal models of Neuronal Ceroid Lipofuscinoses, Pompe disease and Niemann-Pick type C.

Although the nature of the biologic material that accumulates in different lysosomal storage disorders varies, many of these pathologies share common clinical features. Typically, these disorders have multi-organ presentations. The onset of the phenotypes varies and even though lysosomal storage disorders are usually not congenital, in most acute cases the manifestations can be present at birth. In many diseases, like Gaucher, Niemann-Pick, MPSs, and other sphingolipidoses, one of the first pathological manifestations is hepatosplenomegaly, often already present at birth. Cardiomyopathies, including cardiomegaly, heart failure and deposition of glycogen in the heart valves are associated with many lysosomal storage disorders and can be present in newborns, like in infantile Pompe disease, or have a later onset as in several sphingolipidoses. Severe respiratory manifestations have been described in Pompe disease patients, where muscular hypotonicity causes reduction in lung volume; as well as in NPC-2 and Farber disease patients.

Haematological and endocrine manifestations are also typical of lysosomal storage disorders: anaemia and thrombocytopenia are haematological features characteristic of Gaucher disease, while osteopenia and enlargement of endocrine glands are present in other lysosomal storage disorders, especially in MPSs patients. As secondary manifestation of haematological disorders and organomegaly, many lysosomal storage disease patients, including MPS, GM1-gangliosidosis, NP-C, Gaucher and Farber disease, present with hydrops fetalis. Abnormal bone formation, joint contractures and swelling usually develop later in Gaucher, Farber, MPS and GM1-gangliosidosis patients, although bone disease has been occasionally described in neonates. Various cutaneous manifestations, such as ichtchyosis, skin lesions and an increase in body hair are typical of Gaucher, MPSs and Fabry disease. New born patients can also present dysmorphic features, as coarse facies, depressed or absent nasal septum and unusual facial appearances.

The central and peripheral nervous systems are affected in many forms of lysosomal storage diseases, causing a variety of symptoms, including neurocognitive impairment, movement disorders, seizures, optical manifestations and deafness, which usually lead to premature death.

Enzyme replacement therapy is today's standard approved treatment for many lysosomal storage disorders, including Gaucher disease type I, Fabry disease, Pompe disease and some MPSs. Although enzyme replacement therapy is safe and usually well tolerated, it presents some disadvantages: patients are subjected to continuous and frequent infusions; the cost of repetitive administrations is significant; often combination therapies, like bone marrow transplantation are required; and more importantly the currently approved products do not show any efficacy in the treatment of central nervous system pathologies. In fact, the infused recombinant enzyme is not able to cross the blood-brain barrier, even when administered at high dose.

An alternative approach is to use a small molecule drug that reduces the synthesis of the accumulating pathogenic substrate. This is known as substrate reduction therapy. An approved substrate reduction therapy consists of the administration of the imino sugar N-butyldeoxynoj irimycin (miglustat), a competitive inhibitor of ceramide glucosyltransferase that blocks the biosynthesis of glucosylceramide and glucosylceramide-derived glycosphingolipids.

Although miglustat was first commercialised for Gaucher disease type I, it also has potential for treatment of other lysosomal storage disorders, such as Niemann-Pick type C, Fabry disease, and GM1 and GM2-gangliosidose, where secondary accumulation of glucosylceramide-based glycosphingolipids occurs. Moreover, miglustat has shown the ability to cross the blood-brain barrier and therefore it can be used as a treatment for neurological manifestations. The main side effect of miglustat medication is the development of severe gastrointestinal symptoms and occasional peripheral neuropathy and tremor.

Most lysosomal storage disorders are associated with mutations in genes that influence protein conformation, folding and trafficking resulting in unstable and degradable enzymes. Pharmacological chaperones are molecules that, binding to the nascent polypeptides, promote protein stability and inhibit mis-folding and protein aggregation. Pharmacological chaperone therapy had first been proposed as a treatment for Fabry disease, where 1-deoxygalactonojirimycin (migalastat hydrochloride) binds to the active site of a-galactosidase A, increasing its activity. More recently, Orphan Drug designation was granted to Arimoclomol© (Orphazyme AsP) as a potential treatment for Niemann-Pick type C patients. Arimoclomol© is a co-inducer of the heat-shock response that induces the expression of molecular chaperones like Hsp70, and activates natural cellular repair pathways. The treatment has already shown beneficial effects in pre-clinical studies on animal models of amyotrophic lateral sclerosis, spinal bulbar muscular atrophy and retinitis pigmentosa. The on-going phase 2 study (NCT02612129) is currently investigating the efficacy and safety of the drug on NP-C subjects. Since not all mutations will be responsive to potential chaperone therapy and the effects of the treatment may not always be sufficient, researchers are investigating the possibility of chaperone therapy in combination with other treatments.

### Niemann-Pick Disease Type C (NPC)

NP-C patients generally present with neurological degeneration and hepatosplenomegaly (enlargement of liver and spleen) in early childhood, although other clinical phenotypes are well-recognized. The classical presentation of NP-C, which is normally diagnosed in school-age children, consists of ataxia, vertical supranuclear gaze palsy (VSGP), gelastic cataplexy and intellectual regression. Seizures are common and neurological symptoms become disabling. NP-C is caused by mutations in the NPC1 gene. NPC1 encodes the 13 transmembrane domain NPC1 protein, which is localized to the limiting membrane of late endosomes and lysosomes. The function of NPC1 is currently unknown but mutations in the NPC1 gene lead to the accumulation of a variety of lipids in late endosomes/lysosomes (Lloyd-Evans E., et al 2008, Te Vruchte D., et al. 2004). These include cholesterol, glycosphingolipids (GSLs), sphingomyelin and sphingosine, although which of these individually or in concert cause the individual pathological manifestations of this disease is poorly understood (Lloyd-Evans E. and Platt F. M. 2010).

### Expression Constructs of the Invention

An expression construct may be defined as a polynucleotide sequence capable of driving protein expression from a polynucleotide sequence containing a coding sequence. The expression constructs of the present invention comprise NPC1 promoter fragments and human NPC1 (hNPC1). The sequence of NPC1 used in the expression constructs of the present invention is preferably either that of SEQ ID NO: 2 or a hNPC1 nucleotide sequence encoding the polypeptide of SEQ ID NO:3. The sequence ofNPC1 used in the expression constructs of the present invention is preferably either that of SEQ ID NO: 4 or a hNPC1 nucleotide sequence encoding the polypeptide of SEQ ID NO:5. The promoter used in the expression constructs of the present invention is a NPC1 promoter fragment, consisting of a nucleic acid sequence of no more than 400 nucleotides in length. The sequence of the NPC1 promoter used in the present invention is preferably that of SEQ ID NO: 1.

The NPC1 promoter fragment of the present invention may be no more than 400, 390, 380, 370, 360, 350, 340, 330, 320, 310, 309, 308 or 307 nucleotides in length. The NPC1 promoter fragment of the present invention is no more than 400 nucleotides in length and comprises at least 250, 260, 270, 280, 290, 300, 305 consecutive nt from SEQ ID NO:1, or comprises all of SEQ ID NO:1. The NPC1 promoter fragment of the present invention may be no more than 380 nucleotides in length and comprises at least 250, 260, 270, 280, 290, 300, 305 consecutive nt from SEQ ID NO:1, or comprises all of SEQ ID NO:1. The NPC1 promoter fragment of the present invention may be no more than 360 nucleotides in length and comprises at least 250, 260, 270, 280, 290, 300, 305 consecutive nt from SEQ ID NO:1, or comprises all of SEQ ID NO:1. The NPC1 promoter fragment of the present invention may be no more than 350 nucleotides in length and comprises at least 250, 260, 270, 280, 290, 300, 305 consecutive nt from SEQ ID NO:1, or comprises all of SEQ ID NO:1. The NPC1 promoter fragment of the present invention may be no more than 340 nucleotides in length and comprises at least 250, 260, 270, 280, 290, 300, 305 consecutive nt from SEQ ID NO:1, or comprises all of SEQ ID NO:1. The NPC1 promoter fragment of the present invention may be no more than 330 nucleotides in length and comprises at least 250, 260, 270, 280, 290, 300, 305 consecutive nt from SEQ ID NO:1, or comprises all of SEQ ID NO:1. The NPC1 promoter fragment of the present invention may be no more than 320 nucleotides in length and comprises at least 250, 260, 270, 280, 290, 300, 305 consecutive nt from SEQ ID NO:1, or comprises all of SEQ ID NO:1. The NPC1 promoter fragment of the present invention may be no more than 310 nucleotides in length and comprises at least 250, 260, 270, 280, 290, 300, 305 consecutive nt from SEQ ID NO:1, or comprises all of SEQ ID NO:1. The NPC1 promoter fragment of the present invention may consist of SEQ ID NO:1.

The NPC1 promoter for use in the present invention is operably linked to NPC1. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the expression construct.

An expression constructs of the present invention may also include additional nucleotide sequences not naturally found in the NPC1 promoter region or NPC1. An expression construct of the present invention may also include additional nucleotide sequences 5′ to the NPC1 promoter fragment sequence, 3′ to the NPC1 promoter fragment sequence but 5′ to NPC1, and/or 3′ to NPC1.

The expression constructs of the present invention can also be used in tandem with other regulatory elements such as one or more further promoters or enhancers or locus control regions (LCRs).

Vectors of the invention may also incorporate codon-optimised sequences encoding a NPC1 polypeptide. These can be synthesised and incorporated into vectors of the invention using techniques described herein and/or known in the art.

Further expression constructs of the invention may comprise promoters that differ in sequence from the NPC1 promoter fragment sequence above but retain the ability to express NPC1 in cells. Such sequences have at least 75%, at least 80%, at least 85%, at least 90%, at least 95%, at least 98% or at least 99% sequence identity to a sequence of contiguous nucleotides from SEQ ID NO: 1.

Percentage sequence identity of variants is preferably measured over the full length of SEQ ID NO: 1, or over a 200, 210, 220, 230, 240, 250, 260, 270, 280, 290, 300 nucleotide section of SEQ ID NO: 1, or all of SEQ ID NO: 1 aligned with the variant sequence.

Such variant sequences may preferably have at least 75%, at least 80%, at least 85%, at least 90%, at least 95%, at least 98% or at least 99% sequence identity to SEQ ID NO: 1.

Retaining the ability to express NPC1 in cells can be measured by any suitable standard technique known to the person skilled in the art, for example, RNA expression levels can be measured by quantitative real-time PCR. Protein expression can be measured by western blotting or immunohistochemistry.

The expression construct of the invention may comprise the NPC1 nucleotide sequence of SEQ ID NO: 2, or a sequence that encodes the NPC1 sequence of SEQ ID NO: 3, or variants thereof. Further expression constructs of the invention comprise variants of NPC1 that retain the functionality of NPC1. A variant of NPC1 may be defined as any variant of the sequence of SEQ ID NO: 2, including naturally occurring variants in the nucleic acid sequence. The variant may be defined as having at least about 70%, 80%, 90%, 95%, 96%, 97%, 98% or 99% sequence identity to SEQ ID NO: 2, wherein the polypeptide translated from the variant sequence retains its functionality. Preferably, such variant sequences having at least 70%, 80%, 90%, 95%, 96%, 97%, 98% or 99% sequence identity to SEQ ID NO: 2 encode the polypeptide of SEQ ID NO: 3 or a polypeptide having at least 90%, 95%, 98% or 99% identity to SEQ ID NO: 3. The NPC1 nucleotide sequence of SEQ ID NO: 2 encodes the NPC1 sequence of SEQ ID NO: 3.

Further expression constructs of the invention comprise variants of NPC1 that encode the NPC1 polypeptide of SEQ ID NO: 3 and retain the functionality of NPC1. Such a variant may be any sequence encoding SEQ ID NO: 3, including naturally occurring variants in the nucleic acid sequence and optimised sequences.

The expression construct of the invention may comprise the NPC1 nucleotide sequence of SEQ ID NO: 4, or a sequence that encodes the NPC1 sequence of SEQ ID NO: 5, or variants thereof. Further expression constructs of the invention comprise variants of NPC1 that retain the functionality of NPC1. A variant of NPC1 may be defined as any variant of the sequence of SEQ ID NO: 4, including naturally occurring variants in the nucleic acid sequence. The variant may be defined as having at least about 70%, 80%, 90%, 95%, 96%, 97%, 98% or 99% sequence identity to SEQ ID NO: 4, wherein the polypeptide translated from the variant sequence retains its functionality. Preferably, such variant sequences having at least 70%, 80%, 90%, 95%, 96%, 97%, 98% or 99% sequence identity to SEQ ID NO: 4 encode the polypeptide of SEQ ID NO: 5 or a polypeptide having at least 90%, 95%, 98% or 99% identity to SEQ ID NO: 5.

Further expression constructs of the invention comprise variants of NPC1 that encode the NPC1 polypeptide of SEQ ID NO: 5 and retain the functionality of NPC1. Such a variant may be any sequence encoding SEQ ID NO: 5, including naturally occurring variants in the nucleic acid sequence and optimised sequences. The NPC1 nucleotide sequence of SEQ ID NO: 4 encodes the NPC1 sequence of SEQ ID NO: 5.

Other variants may be defined as sequences encoding a polypeptide having at least about 80%, 90%, 95%, 96%, 97%, 98% or 99% sequence identity to the sequence of SEQ ID NO: 3 or 5, wherein the polypeptide translated from the variant sequence retains its functionality.

Retaining NPC1 functionality can be defined as rescuing at least about 50%, 60%, 70%, 80% 90%, 95%, 96%, 97%, 98%, 99% or 100% of NPC1 function.

NPC1 function can be analysed by any suitable standard technique known to the person skilled in the art. Assays that focus on assessing the correction of downstream pathology, for example a reduction in esterified cholesterol accumulation via filipin staining, reduction of glycosphingolipid accumulation via normal phase high-performance liquid chromatography or reduction in lysosomal size/number via lysotracker can be used to assess NPC1 function. Alternatively function can also be assessed indirectly via in vivo delivery of an NPC1 gene product to be tested and monitoring for therapeutic efficacy via weight loss, survival, behavioural analysis and immunohistochemistry.

“Codon optimization” relates to the process of altering a naturally occurring polynucleotide sequence to enhance expression in the target organism, for example, humans. In one embodiment of the present invention, NPC1 is codon optimised.

Sequence identity may be calculated using any suitable algorithm. For example the PILEUP and BLAST algorithms can be used to calculate identity or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate identity (for example used on its default settings) (Devereux et al (1984) Nucleic Acids Research 12, 387-395).

The expression constructs of the present invention can be used to drive significantly increased expression of NPC1 in cells. Significant increased expression can be defined as more than about 10 times, 20 times, 50 times, 100 times, 200 times or 300 times the expression of NPC1 in cells when compared with wild-type expression of NPC1. Expression of NPC1 can be measured by any suitable standard technique known to the person skilled in the art. For example, RNA expression levels can be measured by quantitative real-time PCR. Protein expression can be measured by Western blotting or immunohistochemistry.

Vectors

The present invention provides vectors comprising the expression constructs of the present invention. The vector may be of any type, for example it may be a plasmid vector or a minicircle DNA.

Typically, vectors of the invention are however viral vectors. The viral vector may be based on the herpes simplex virus, adenovirus or lentivirus. The viral vector may be an adeno-associated virus (AAV) vector or a derivative thereof.

The viral vector derivative may be a chimeric, shuffled or capsid modified derivative.

The viral vector may comprise an AAV genome from a naturally derived serotype, isolate or Glade of AAV.

The serotype may for example be AAV2, AAVS or AAV8.

The efficacy of gene therapy is, in general, dependent upon adequate and efficient delivery of the donated DNA. This process is usually mediated by viral vectors. Adeno-associated viruses (AAV), a member of the parvovirus family, are commonly used in gene therapy. Wild-type AAV, containing viral genes, insert their genomic material into chromosome 19 of the host cell (Kotin, et al. 1990). The AAV single-stranded DNA genome comprises two inverted terminal repeats (ITRs) and two open reading frames, containing structural (cap) and packaging (rep) genes (Hermonat et al. 1984).

For therapeutic purposes, the only sequences required in cis, in addition to the therapeutic gene, are the ITRs. The AAV virus is therefore modified: the viral genes are removed from the genome, producing recombinant AAV (rAAV). This contains only the therapeutic gene, the two ITRs. The removal of the viral genes renders rAAV incapable of actively inserting its genome into the host cell DNA. Instead, the rAAV genomes fuse via the ITRs, forming circular, episomal structures, or insert into pre-existing chromosomal breaks. For viral production, the structural and packaging genes, now removed from the rAAV, are supplied in trans, in the form of a helper plasmid.

AAV is a particularly attractive vector as it is generally non-pathogenic; the majority people have been infected with this virus during their life with no adverse effects (Erles et al. 1999). Despite this, there are several drawbacks to the use of rAAV in gene therapy, although the majority of these only apply to systemic administration of rAAV. Nevertheless, it is important to acknowledge these potential limitations. Infection can trigger the following immunological responses:

As the majority of the human population is seropositive for AAV, neutralising antibodies against rAAV can impair gene delivery (Moskalenko et al. 2000; Sun et al. 2003).

Systemically delivered rAAV can trigger a capsid protein-directed T-cell response, leading to the apoptosis of transduced cells (Manno et al. 2006).

rAAV vectors can trigger complement activation (Zaiss et al. 2008).

As the rAAV delivery is generally unspecific, the vector can accumulate in the liver (Michelfelder et al. 2009).

AAV vectors are limited by a relatively small packaging capacity of roughly 4.8 kb and a slow onset of expression following transduction (Dong et al. 1996).

Most vector constructs are based on the AAV serotype 2 (AAV2). AAV2 binds to the target cells via the heparin sulphate proteoglycan receptor (Summerford and and Samulski 1998). The AAV2 genome, like those of all AAV serotypes, can be enclosed in a number of different capsid proteins. AAV2 can be packaged in its natural AAV2 capsid (AAV2/2) or it can be pseudotyped with other capsids (e.g. AAV2 genome in AAV1 capsid; AAV2/1, AAV2 genome in AAVS capsid; AAV2/5 and AAV2 genome in AAV8 capsid; AAV2/8).

rAAV transduces cells via serotype specific receptor-mediated endocytosis. A major factor influencing the kinetics of rAAV transgene expression is the rate of virus particle uncoating within the endosome (Thomas et al. 2004). This, in turn, depends upon the type of capsid enclosing the genetic material (Ibid.). After uncoating the linear single-stranded rAAV genome is stabilised by forming a double-stranded molecule via de novo synthesis of a complementary strand (Vincent-Lacaze et al. 1999). The use of self-complementary DNA may bypass this stage by producing double-stranded transgene DNA. It has been shown that self-complementary AAV2/8 gene expression is of faster onset and higher amplitude, compared to single-stranded AAV2/8. Thus, by circumventing the time lag associated with second-strand synthesis, gene expression levels are increased, when compared to transgene expression from standard single-stranded constructs. Subsequent studies investigating the effect of self-complementary DNA in other AAV pseudotypes (e.g. AAV2/5) have produced similar results . One caveat to this technique is that, as AAV has a packaging capacity of approximately 4.8 kb, the self-complementary recombinant genome must be appropriately sized (i.e. 2.3 kb or less).

In addition to modifying packaging capacity, pseudotyping the AAV2 genome with other AAV capsids can alter cell specificity and the kinetics of transgene expression.

### AAV Genome

The vector of the present invention may comprise an adeno-associated virus (AAV) genome or a derivative thereof.

An AAV genome is a polynucleotide sequence which encodes functions needed for production of an AAV viral particle. These functions include those operating in the replication and packaging cycle for AAV in a host cell, including encapsidation of the AAV genome into an AAV viral particle. Naturally occurring AAV viruses are replication-deficient and rely on the provision of helper functions in trans for completion of a replication and packaging cycle. Accordingly and with the additional removal of the AAV rep and cap genes, the AAV genome of the vector of the invention is replication-deficient.

The AAV genome may be in single-stranded form, either positive or negative-sense, or alternatively in double-stranded form. The use of a double-stranded form allows bypass of the DNA replication step in the target cell and so can accelerate transgene expression.

The AAV genome may be from any naturally derived serotype or isolate or Glade of AAV. As is known to the skilled person, AAV viruses occurring in nature may be classified according to various biological systems.

Commonly, AAV viruses are referred to in terms of their serotype. A serotype corresponds to a variant subspecies of AAV which owing to its profile of expression of capsid surface antigens has a distinctive reactivity which can be used to distinguish it from other variant subspecies. Typically, a virus having a particular AAV serotype does not efficiently cross-react with neutralising antibodies specific for any other AAV serotype. AAV serotypes include AAV1, AAV2, AAV3, AAV4, AAVS, AAV6, AAV7, AAV8, AAV9, AAV10 and AAV11, also recombinant serotypes, such as Rec2 and Rec3, recently identified from primate brain. In vectors of the invention, the genome may be derived from any AAV serotype. The capsid may also be derived from any AAV serotype. The genome and the capsid may be derived from the same serotype or different serotypes.

In vectors of the invention, it is preferred that the genome is derived from AAV serotype 2 (AAV2), AAV serotype 4 (AAV4), AAV serotype 5 (AAV5) or AAV serotype 8 (AAV8). It is most preferred that the genome is derived from AAV2 but other serotypes of particular interest for use in the invention include AAV4, AAV5 and AAV8. It is preferred that the capsid is derived from AAV9.

In a preferred embodiment of the invention the genome is derived from AAV serotype 2 (AAV2) and the capsid is derived from AAV9, i.e. AAV2/9.

Reviews of AAV serotypes may be found in Choi et al (Curr Gene Ther. 2005; 5(3); 299-310) and Wu et al (Molecular Therapy. 2006; 14(3), 316-327). The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV viruses may also be referred to in terms of clades or clones. This refers to the phylogenetic relationship of naturally derived AAV viruses, and typically to a phylogenetic group of AAV viruses which can be traced back to a common ancestor, and includes all descendants thereof. Additionally, AAV viruses may be referred to in terms of a specific isolate, i.e. a genetic isolate of a specific AAV virus found in nature. The term genetic isolate describes a population of AAV viruses which has undergone limited genetic mixing with other naturally occurring AAV viruses, thereby defining a recognisably distinct population at a genetic level.

Examples of clades and isolates of AAV that may be used in the invention include:

Clade A: AAV1 NC_002077, AF063497, AAV6 NC_001862, Hu. 48 AY530611, Hu 43 AY530606, Hu 44 AY530607, Hu 46 AY530609

Clade B: Hu. 19 AY530584, Hu. 20 AY530586, Hu 23 AY530589, Hu22 AY530588, Hu24 AY530590, Hu21 AY530587, Hu27 AY530592, Hu28 AY530593, Hu 29 AY530594, Hu63 AY530624, Hu64 AY530625, Hu13 AY530578, Hu56 AY530618, Hu57 AY530619, Hu49 AY530612, Hu58 AY530620, Hu34 AY530598, Hu35 AY530599, AAV2 NC_001401, Hu45 AY530608, Hu47 AY530610, Hu51 AY530613, Hu52 AY530614, Hu T41 AY695378, Hu S17 AY695376, Hu T88 AY695375, Hu T71 AY695374, Hu T70 AY695373, Hu T40 AY695372, Hu T32 AY695371, Hu T17 AY695370, Hu LG15 AY695377,

Clade C: Hu9 AY530629, Hu10 AY530576, Hu11 AY530577, Hu53 AY530615, Hu55 AY530617, Hu54 AY530616, Hu7 AY530628, Hu18 AY530583, Hu15 AY530580, Hu16 AY530581, Hu25 AY530591, Hu60 AY530622, Ch5 AY243021, Hu3 AY530595, Hu1 AY530575, Hu4 AY530602 Hu2, AY530585, Hu61 AY530623

Clade D: Rh62 AY530573, Rh48 AY530561, Rh54 AY530567, Rh55 AY530568, Cy2 AY243020, AAV7 AF513851, Rh35 AY243000, Rh37 AY242998, Rh36 AY242999, Cy6 AY243016, Cy4 AY243018, Cy3 AY243019, Cy5 AY243017, Rh13 AY243013

Clade E: Rh38 AY530558, Hu66 AY530626, Hu42 AY530605, Hu67 AY530627, Hu40 AY530603, Hu41 AY530604, Hu37 AY530600, Rh40 AY530559, Rh2 AY243007, Bb1 AY243023, Bb2 AY243022, Rh10 AY243015, Hu11 AY530582, Hu6 AY530621, Rh25 AY530557, Pi2 AY530554, Pil AY530553, Pi3 AY530555, Rh57 AY530569, Rh50 AY530563, Rh49 AY530562, Hu39 AY530601, Rh58 AY530570, Rh61 AY530572, Rh52 AY530565, Rh53 AY530566, Rh51 AY530564, Rh64 AY530574, Rh43 AY530560, AAV8 AF513852, Rh8 AY242997, Rhl AY530556

Clade F: Hu14 (AAV9) AY530579, Hu31 AY530596, Hu32 AY530597, Clonal Isolate AAVS Y18065, AF085716, AAV 3 NC_001729, AAV 3B NC_001863, AAV4 NC_001829, Rh34 AY243001, Rh33 AY243002, Rh32 AY243003/

The skilled person can select an appropriate serotype, Glade, clone or isolate of AAV for use in the present invention on the basis of their common general knowledge.

It should be understood however that the invention also encompasses use of an AAV genome of other serotypes that may not yet have been identified or characterised. The AAV serotype determines the tissue specificity of infection (or tropism) of an AAV virus.

Typically, the AAV genome of a naturally derived serotype or isolate or Glade of AAV comprises at least one inverted terminal repeat sequence (ITR). Vectors of the invention typically comprise two ITRs, preferably one at each end of the genome. An ITR sequence acts in cis to provide a functional origin of replication, and allows for integration and excision of the vector from the genome of a cell. Preferred ITR sequences are those of AAV2 and variants thereof. The AAV genome typically comprises packaging genes, such as rep and/or cap genes which encode packaging functions for an AAV viral particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV viral particle. Capsid variants are discussed below.

Preferably the AAV genome will be derivatised for the purpose of administration to patients. Such derivatisation is standard in the art and the present invention encompasses the use of any known derivative of an AAV genome, and derivatives which could be generated by applying techniques known in the art. Derivatisation of the AAV genome and of the AAV capsid are reviewed in Coura and Nardi (Virology Journal, 2007, 4:99), and in Choi et al and Wu et al, referenced above.

Derivatives of an AAV genome include any truncated or modified forms of an AAV genome which allow for expression of a Rep-1 transgene from a vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences i.e. a self-complementary AAV genome. This allows for bypass of DNA replication in the target cell, and so enables accelerated transgene expression.

The one or more ITRs will preferably flank the expression construct cassette containing the promoter and transgene of the invention. The inclusion of one or more ITRs is preferred to aid packaging of the vector of the invention into viral particles. In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

With reference to the AAV2 genome, the following portions could therefore be removed in a derivative of the invention: One inverted terminal repeat (ITR) sequence, the replication (rep) and capsid (cap) genes. However, in some embodiments, including in vitro embodiments, derivatives may additionally include one or more rep and/or cap genes or other viral sequences of an AAV genome.

A derivative may be a chimeric, shuffled or capsid-modified derivative of one or more naturally occurring AAV viruses. The invention encompasses the provision of capsid protein sequences from different serotypes, clades, clones, or isolates of AAV within the same vector. The invention encompasses the packaging of the genome of one serotype into the capsid of another serotype i.e. pseudotyping.

Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV viral vector comprising a naturally occurring AAV genome, such as that of AAV2. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are cotransfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N-and/or C-terminus of a capsid coding sequence.

The unrelated protein or peptide may advantageously be one which acts as a ligand for a particular cell type, thereby conferring improved binding to a target cell or improving the specificity of targeting of the vector to a particular cell population.

The unrelated protein may also be one which assists purification of the viral particle as part of the production process i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al, referenced above.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The vector of the invention takes the form of a viral vector comprising the expression constructs of the invention.

For the avoidance of doubt, the invention also provides an AAV viral particle comprising a vector of the invention. The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral envelope. The AAV particle also includes chemically modified forms bearing ligands adsorbed to the capsid surface. For example, such ligands may include antibodies for targeting a particular cell surface receptor.

The invention additionally provides a host cell comprising a vector or AAV viral particle of the invention.

Preparation of Vector

The vector of the invention may be prepared by standard means known in the art for provision of vectors for gene therapy. Thus, well established public domain transfection, packaging and purification methods can be used to prepare a suitable vector preparation.

As discussed above, a vector of the invention may comprise the full genome of a naturally occurring AAV virus in addition to a promoter of the invention or a variant thereof. However, commonly a derivatised genome will be used, for instance a derivative which has at least one inverted terminal repeat sequence (ITR), but which may lack any AAV genes such as rep or cap.

In such embodiments, in order to provide for assembly of the derivatised genome into an AAV viral particle, additional genetic constructs providing AAV and/or helper virus functions will be provided in a host cell in combination with the derivatised genome. These additional constructs will typically contain genes encoding structural AAV capsid proteins i.e. cap, VP1, VP2, VP3, and genes encoding other functions required for the AAV life cycle, such as rep. The selection of structural capsid proteins provided on the additional construct will determine the serotype of the packaged viral vector.

A particularly preferred packaged viral vector for use in the invention comprises a derivatised genome of AAV2 in combination with AAV9 capsid proteins.

As mentioned above, AAV viruses are replication incompetent and so helper virus functions, preferably adenovirus helper functions will typically also be provided on one or more additional constructs to allow for AAV replication.

All of the above additional constructs may be provided as plasmids or other episomal elements in the host cell, or alternatively one or more constructs may be integrated into the genome of the host cell.

Expression constructs and vectors of the invention have the ability to rescue loss of NPC1 function, which may occur for example by mutations in the NPC1 gene. “Rescue” generally means any amelioration or slowing of progression of a Niemann-Pick disease type C (NPC) disease phenotype, for example restoring the presence of NPC1 protein in the brain, thus ameliorating neuronal pathologies.

The properties of the expression constructs and vectors of the invention can also be tested using techniques known by the person skilled in the art. In particular, a sequence of the invention can be assembled into a vector of the invention and delivered to a NPC1-deficient test animal, such as a mouse, and the effects observed and compared to a control.

### Methods of Therapy and Medical Uses

The expression constructs and vectors of the invention may be used in the treatment or prevention of Niemann-Pick disease type C (NPC) disease.

The expression constructs and vectors of the present invention can also be used in the treatment and/or prevention of diseases that are associated with that loss of NPC1 function.

This provides a means whereby the degenerative process of the diseases can be treated, arrested, palliated or prevented.

The invention therefore provides a pharmaceutical composition comprising the vector of the invention and a pharmaceutically acceptable carrier.

The invention also provides a vector for use in a method of preventing or treating Niemann-Pick disease type C (NPC) disease.

The invention also provides the use of a vector of the invention in the manufacture of a medicament for the treatment or prevention of Niemann-Pick disease type C (NPC) disease.

The invention also provides a method of treating or preventing Niemann-Pick disease type C (NPC) disease in a patient in need thereof comprising administering a therapeutically effective amount of a vector of the invention to the patient.

In a preferred embodiment of the invention, the neurological complications of Niemann-Pick disease type C (NPC) are prevented or treated by use of the expression constructs and vectors of the present invention.

In general, parenteral routes of delivery of vectors of the invention, such as intravenous (IV), or intracisternal magna (ICM) or intracerebroventricular (ICV) administration, typically by injection, are preferred.

The invention therefore also provides a method of treating or preventing Niemann-Pick disease type C (NPC) disease in a patient in need thereof, comprising administering a therapeutically effective amount of a vector of the invention to the patient by a parenteral route of administration. Accordingly, Niemann-Pick disease type C (NPC) disease is thereby treated or prevented in said patient.

In a related aspect, the invention provides for use of a vector of the invention in a method of treating or preventing Niemann-Pick disease type C (NPC) disease by administering said vector to a patient by a parenteral route of administration. Additionally, the invention provides the use of a vector of the invention in the manufacture of a medicament for treating or preventing Niemann-Pick disease type C (NPC) disease by a parenteral route of administration.

In all these embodiments, the vector of the invention may be administered in order to prevent the onset of one or more symptoms of Niemann-Pick disease type C (NPC) disease. The patient may be asymptomatic. The subject may have a predisposition to the disease. The method or use may comprise a step of identifying whether or not a subject is at risk of developing, or has, Niemann-Pick disease type C (NPC) disease.

A prophylactically effective amount of the vector is administered to such a subject. A prophylactically effective amount is an amount which prevents the onset of one or more symptoms of the disease.

Alternatively, the vector may be administered once the symptoms of the disease have appeared in a subject i.e. to cure existing symptoms of the disease. A therapeutically effective amount of the antagonist is administered to such a subject. A therapeutically effective amount is an amount which is effective to ameliorate one or more symptoms of the disease.

The subject may be male or female. The subject is preferably identified as being at risk of, or having, the disease. The administration of the vector is typically by a parenteral route of administration, or a combination of parenteral routes of administration. Parenteral routes of administration encompass intravenous (IV), intracisternal magna (ICM), intramuscular (IM), subcutaneous (SC), epidural (E), intracerebral (IC), intracerebroventricular (ICV) and intradermal (ID) administration.

The dose of a vector of the invention may be determined according to various parameters, especially according to the age, weight and condition of the patient to be treated; the route of administration; and the required regimen. Again, a physician will be able to determine the required route of administration and dosage for any particular patient. For example, a suitable dose of a vector of the present invention for IV administration may be in the range of 6.7×10¹³ vg/kg to 2.0×10¹⁴ vg/kg, where vg=viral genome. For ICV administration total dose could range from 1.0×10¹° vg/kg to 2.0×10¹⁴ vg/kg.

The dose may be provided as a single dose, but may be repeated in cases where vector may not have targeted the correct region. The treatment is preferably a single permanent approach, but repeat injections, for example in future years and/or with different AAV serotypes may be considered.

### Host Cells

Any suitable host cell can be used to produce the vectors of the invention. In general, such cells will be transfected mammalian cells but other cell types, e.g. insect cells, can also be used. In terms of mammalian cell production systems, HEK293 and HEK293T are preferred for AAV vectors. BHK or CHO cells may also be used.

### Pharmaceutical Compositions and Dosages

The vector of the invention can be formulated into pharmaceutical compositions. These compositions may comprise, in addition to the vector, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may be determined by the skilled person according to the route of administration.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used.

For injection at the site of affliction, the active ingredient will be in the form of an aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection, Hartmann's solution. Preservatives, stabilisers, buffers, antioxidants and/or other additives may be included, as required.

For delayed release, the vector may be included in a pharmaceutical composition which is formulated for slow release, such as in microcapsules formed from biocompatible polymers or in liposomal carrier systems according to methods known in the art.

Dosages and dosage regimes can be determined within the normal skill of the medical practitioner responsible for administration of the composition.

### Combination Therapies

The expression constructs, vectors and/or pharmaceutical compositions can be used in combination with any other therapy for the treatment or prevention of Niemann-Pick disease type C (NPC) disease, such as the substrate reduction therapy (SRT) miglustat or the chaperone therapy Arimoclomol.

### Kits

The expression constructs, vectors and/or pharmaceutical compositions can be packaged into a kit.

### EXAMPLES

Sequences
SEQ ID NO: 1-307 bp optimised NPC1 promoter sequence
CGAGCCAGACTCCATAAGTCCCGCGCCTGGCCCCCGGGGATTGCAGGGGCTGAGGAGAAGGGCAACACGGGGACC
TTGAAGCGGGGTCGCGGCGGCGCCCCAGCCCGGGCCAGGGAGTCCCGGCAGCGGCACCTCCCAGAAAGGGCGGAG
CCGACGACGCCTTCTTCCTTCCTGACCGGCGCGCGCAGCCTGCTGCCGCGGTCAGCGCCTGCTCCTGCTCCTCCG
CTCCTCCTGCGCGGGGTGCTGAAACAGCCCGGGGAAGTAGAGCCGCCTCCGGGGAGCCCAACCAGCCGAACGCCG
CCGGCGT
SEQ ID NO: 2-human NPC1 nucleotide sequence-NCBI Reference Sequence:
NM_000271.5
CTTCCTGACCGGCGCGCGCAGCCTGCTGCCGCGGTCAGCGCCTGCTCCTGCTCCTCCGCTCCTCCTGCGCGGGGT
GCTGAAACAGCCCGGGGAAGTAGAGCCGCCTCCGGGGAGCCCAACCAGCCGAACGCCGCCGGCGTCAGCAGCCTT
GCGCGGCCACAGCATGACCGCTCGCGGCCTGGCCCTTGGCCTCCTCCTGCTGCTACTGTGTCCAGCGCAGGTGTT
TTCACAGTCCTGTGTTTGGTATGGAGAGTGTGGAATTGCATATGGGGACAAGAGGTACAATTGCGAATATTCTGG
CCCACCAAAACCATTGCCAAAGGATGGATATGACTTAGTGCAGGAACTCTGTCCAGGATTCTTCTTTGGCAATGT
CAGTCTCTGTTGTGATGTTCGGCAGCTTCAGACACTAAAAGACAACCTGCAGCTGCCTCTACAGTTTCTGTCCAG
ATGTCCATCCTGTTTTTATAACCTACTGAACCTGTTTTGTGAGCTGACATGTAGCCCTCGACAGAGTCAGTTTTT
GAATGTTACAGCTACTGAAGATTATGTTGATCCTGTTACAAACCAGACGAAAACAAATGTGAAAGAGTTACAATA
CTACGTCGGACAGAGTTTTGCCAATGCAATGTACAATGCCTGCCGGGATGTGGAGGCCCCCTCAAGTAATGACAA
GGCCCTGGGACTCCTGTGTGGGAAGGACGCTGACGCCTGTAATGCCACCAACTGGATTGAATACATGTTCAATAA
GGACAATGGACAGGCACCTTTTACCATCACTCCTGTGTTTTCAGATTTTCCAGTCCATGGGATGGAGCCCATGAA
CAATGCCACCAAAGGCTGTGACGAGTCTGTGGATGAGGTCACAGCACCATGTAGCTGCCAAGACTGCTCTATTGT
CTGTGGCCCCAAGCCCCAGCCCCCACCTCCTCCTGCTCCCTGGACGATCCTTGGCTTGGACGCCATGTATGTCAT
CATGTGGATCACCTACATGGCGTTTTTGCTTGTGTTTTTTGGAGCATTTTTTGCAGTGTGGTGCTACAGAAAACG
GTATTTTGTCTCCGAGTACACTCCCATCGATAGCAATATAGCTTTTTCTGTTAATGCAAGTGACAAAGGAGAGGC
GTCCTGCTGTGACCCTGTCAGCGCAGCATTTGAGGGCTGCTTGAGGCGGCTGTTCACACGCTGGGGGTCTTTCTG
CGTCCGAAACCCTGGCTGTGTCATTTTCTTCTCGCTGGTCTTCATTACTGCGTGTTCGTCAGGCCTGGTGTTTGT
CCGGGTCACAACCAATCCAGTTGACCTCTGGTCAGCCCCCAGCAGCCAGGCTCGCCTGGAAAAAGAGTACTTTGA
CCAGCACTTTGGGCCTTTCTTCCGGACGGAGCAGCTCATCATCCGGGCCCCTCTCACTGACAAACACATTTACCA
GCCATACCCTTCGGGAGCTGATGTACCCTTTGGACCTCCGCTTGACATACAGATACTGCACCAGGTTCTTGACTT
ACAAATAGCCATCGAAAACATTACTGCCTCTTATGACAATGAGACTGTGACACTTCAAGACATCTGCTTGGCCCC
TCTTTCACCGTATAACACGAACTGCACCATTTTGAGTGTGTTAAATTACTTCCAGAACAGCCATTCCGTGCTGGA
CCACAAGAAAGGGGACGACTTCTTTGTGTATGCCGATTACCACACGCACTTTCTGTACTGCGTACGGGCTCCTGC
CTCTCTGAATGATACAAGTTTGCTCCATGACCCTTGTCTGGGTACGTTTGGTGGACCAGTGTTCCCGTGGCTTGT
GTTGGGAGGCTATGATGATCAAAACTACAATAACGCCACTGCCCTTGTGATTACCTTCCCTGTCAATAATTACTA
TAATGATACAGAGAAGCTCCAGAGGGCCCAGGCCTGGGAAAAAGAGTTTATTAATTTTGTGAAAAACTACAAGAA
TCCCAATCTGACCATTTCCTTCACTGCTGAACGAAGTATTGAAGATGAACTAAATCGTGAAAGTGACAGTGATGT
CTTCACCGTTGTAATTAGCTATGCCATCATGTTTCTATATATTTCCCTAGCCTTGGGGCACATGAAAAGCTGTCG
CAGGCTTCTGGTGGATTCGAAGGTCTCACTAGGCATCGCGGGCATCTTGATCGTGCTGAGCTCGGTGGCTTGCTC
CTTGGGTGTCTTCAGCTACATTGGGTTGCCCTTGACCCTCATTGTGATTGAAGTCATCCCGTTCCTGGTGCTGGC
TGTTGGAGTGGACAACATCTTCATTCTGGTGCAGGCCTACCAGAGAGATGAACGTCTTCAAGGGGAAACCCTGGA
TCAGCAGCTGGGCAGGGTCCTAGGAGAAGTGGCTCCCAGTATGTTCCTGTCATCCTTTTCTGAGACTGTAGCATT
TTTCTTAGGAGCATTGTCCGTGATGCCAGCCGTGCACACCTTCTCTCTCTTTGCGGGATTGGCAGTCTTCATTGA
CTTTCTTCTGCAGATTACCTGTTTCGTGAGTCTCTTGGGGTTAGACATTAAACGTCAAGAGAAAAATCGGCTAGA
CATCTTTTGCTGTGTCAGAGGTGCTGAAGATGGAACAAGCGTCCAGGCCTCAGAGAGCTGTTTGTTTCGCTTCTT
CAAAAACTCCTATTCTCCACTTCTGCTAAAGGACTGGATGAGACCAATTGTGATAGCAATATTTGTGGGTGTTCT
GTCATTCAGCATCGCAGTCCTGAACAAAGTAGATATTGGATTGGATCAGTCTCTTTCGATGCCAGATGACTCCTA
CATGGTGGATTATTTCAAATCCATCAGTCAGTACCTGCATGCGGGTCCGCCTGTGTACTTTGTCCTGGAGGAAGG
GCACGACTACACTTCTTCCAAGGGGCAGAACATGGTGTGCGGCGGCATGGGCTGCAACAATGATTCCCTGGTGCA
GCAGATATTTAACGCGGCGCAGCTGGACAACTATACCCGAATAGGCTTCGCCCCCTCGTCCTGGATCGACGATTA
TTTCGACTGGGTGAAGCCACAGTCGTCTTGCTGTCGAGTGGACAATATCACTGACCAGTTCTGCAATGCTTCAGT
GGTTGACCCTGCCTGCGTTCGCTGCAGGCCTCTGACTCCGGAAGGCAAACAGAGGCCTCAGGGGGGAGACTTCAT
GAGATTCCTGCCCATGTTCCTTTCGGATAACCCTAACCCCAAGTGTGGCAAAGGGGGACATGCTGCCTATAGTTC
TGCAGTTAACATCCTCCTTGGCCATGGCACCAGGGTCGGAGCCACGTACTTCATGACCTACCACACCGTGCTGCA
GACCTCTGCTGACTTTATTGACGCTCTGAAGAAAGCCCGACTTATAGCCAGTAATGTCACCGAAACCATGGGCAT
TAACGGCAGTGCCTACCGAGTATTTCCTTACAGTGTGTTTTATGTCTTCTACGAACAGTACCTGACCATCATTGA
CGACACTATCTTCAACCTCGGTGTGTCCCTGGGCGCGATATTTCTGGTGACCATGGTCCTCCTGGGCTGTGAGCT
CTGGTCTGCAGTCATCATGTGTGCCACCATCGCCATGGTCTTGGTCAACATGTTTGGAGTTATGTGGCTCTGGGG
CATCAGTCTGAACGCTGTATCCTTGGTCAACCTGGTGATGAGCTGTGGCATCTCCGTGGAGTTCTGCAGCCACAT
AACCAGAGCGTTCACGGTGAGCATGAAAGGCAGCCGCGTGGAGCGCGCGGAAGAGGCACTTGCCCACATGGGCAG
CTCCGTGTTCAGTGGAATCACACTTACAAAATTTGGAGGGATTGTGGTGTTGGCTTTTGCCAAATCTCAAATTTT
CCAGATATTCTACTTCAGGATGTATTTGGCCATGGTCTTACTGGGAGCCACTCACGGATTAATATTTCTCCCTGT
CTTACTCAGTTACATAGGGCCATCAGTAAATAAAGCCAAAAGTTGTGCCACTGAAGAGCGATACAAAGGAACAGA
GCGCGAACGGCTTCTAAATTTCTAGCCCTCTCGCAGGGCATCCTGACTGAACTGTGTCTAAGGGTCGGTCGGTTT
ACCACTGGACGGGTGCTGCATCGGCAAGGCCAAGTTGAACACCGGATGGTGCCAACCATCGGTTGTTTGGCAGCA
GCTTTGAACGTAGCGCCTGTGAACTCAGGAATGCACAGTTGACTTGGGAAGCAGTATTACTAGATCTGGAGGCAA
CCACAGGACACTAAACTTCTCCCAGCCTCTTCAGGAAAGAAACCTCATTCTTTGGCAAGCAGGAGGTGACACTAG
ATGGCTGTGAATGTGATCCGCTCACTGACACTCTGTAAAGGCCAATCAATGCACTGTCTGTCTCTCCTTTTAGGA
GTAAGCCATCCCACAAGTTCTATACCATATTTTTAGTGACAGTTGAGGTTGTAGATACACTTTATAACATTTTAT
AGTTTAAAGAGCTTTATTAATGCAATAAATTAACTTTGTACACATTTTTATATAAAAAAACAGCAAGTGATTTCA
GAATGTTGTAGGCCTCATTAGAGCTTGGTCTCCAAAAATCTGTTTGAAAAAAGCAACATGTTCTTCACAGTGTTC
CCCTAGAAAGGAAGAGATTTAATTGCCAGTTAGATGTGGCATGAAATGAGGGACAAAGAAAGCATCTCGTAGGTG
TGTCTACTGGGTTTTAACTTATTTTTCTTTAATAAAATACATTGTTTTCCTAAGTTTTGGGGTTACCCTATCTGC
TTTGAGAGACAAATACAAAAGCTAAATGGAAGAGA
SEQ ID NO: 3-human NPC1 protein sequence-NCBI Reference Sequence:
NP_000262.2
MTARGLALGLLLLLLCPAQVFSQSCVWYGECGIAYGDKRYNCEYSGPPKPLPKDGYDLVQELCPGFFFGNVSLCC
DVRQLQTLKDNLQLPLQFLSRCPSCFYNLLNLFCELTCSPRQSQFLNVTATEDYVDPVTNQTKTNVKELQYYVGQ
SFANAMYNACRDVEAPSSNDKALGLLCGKDADACNATNWIEYMFNKDNGQAPFTITPVFSDFPVHGMEPMNNATK
GCDESVDEVTAPCSCQDCSIVCGPKPQPPPPPAPWTILGLDAMYVIMWITYMAFLLVFFGAFFAVWCYRKRYFVS
EYTPIDSNIAFSVNASDKGEASCCDPVSAAFEGCLRRLFTRWGSFCVRNPGCVIFFSLVFITACSSGLVFVRVTT
NPVDLWSAPSSQARLEKEYFDQHFGPFFRTEQLIIRAPLTDKHIYQPYPSGADVPFGPPLDIQILHQVLDLQIAI
ENITASYDNETVTLQDICLAPLSPYNTNCTILSVLNYFQNSHSVLDHKKGDDFFVYADYHTHFLYCVRAPASLND
TSLLHDPCLGTFGGPVFPWLVLGGYDDQNYNNATALVITFPVNNYYNDTEKLQRAQAWEKEFINFVKNYKNPNLT
ISFTAERSIEDELNRESDSDVFTVVISYAIMFLYISLALGHMKSCRRLLVDSKVSLGIAGILIVLSSVACSLGVF
SYIGLPLTLIVIEVIPFLVLAVGVDNIFILVQAYQRDERLQGETLDQQLGRVLGEVAPSMFLSSFSETVAFFLGA
LSVMPAVHTFSLFAGLAVFIDFLLQITCFVSLLGLDIKRQEKNRLDIFCCVRGAEDGTSVQASESCLFRFFKNSY
SPLLLKDWMRPIVIAIFVGVLSFSIAVLNKVDIGLDQSLSMPDDSYMVDYFKSISQYLHAGPPVYFVLEEGHDYT
SSKGQNMVCGGMGCNNDSLVQQIFNAAQLDNYTRIGFAPSSWIDDYFDWVKPQSSCCRVDNITDQFCNASVVDPA
CVRCRPLTPEGKQRPQGGDFMRFLPMFLSDNPNPKCGKGGHAAYSSAVNILLGHGTRVGATYFMTYHTVLQTSAD
FIDALKKARLIASNVTETMGINGSAYRVFPYSVFYVFYEQYLTIIDDTIFNLGVSLGAIFLVTMVLLGCELWSAV
IMCATIAMVLVNMFGVMWLWGISLNAVSLVNLVMSCGISVEFCSHITRAFTVSMKGSRVERAEEALAHMGSSVFS
GITLTKFGGIVVLAFAKSQIFQIFYFRMYLAMVLLGATHGLIFLPVLLSYIGPSVNKAKSCATEERYKGTERERL
LNF
SEQ ID NO: 4-human NPC1 nucleotide sequence-NCBI Reference Sequence
AF002020.1
TTTGCTCCTGCTCCTCCGCTCCTCCTGCGCGGGGTGCTGAAACAGCCCGGGGAAGTAGAGCCGCCTCCGG
GGAGCCCAACCAGCCGAACGCCGCCGGCGTCAGCAGCCTTGCGCGGCCACAGCATGACCGCTCGCGGCCT
GGCCCTTGGCCTCCTCCTGCTGCTACTGTGTCCAGCGCAGGTGTTTTCACAGTCCTGTGTTTGGTATGGA
GAGTGTGGAATTGCATATGGGGACAAGAGGTACAATTGCGAATATTCTGGCCCACCAAAACCATTGCCAA
AGGATGGATATGACTTAGTGCAGGAACTCTGTCCAGGATTCTTCTTTGGCAATGTCAGTCTCTGTTGTGA
TGTTCGGCAGCTTCAGACACTAAAAGACAACCTGCAGCTGCCTCTACAGTTTCTGTCCAGATGTCCATCC
TGTTTTTATAACCTACTGAACCTGTTTTGTGAGCTGACATGTAGCCCTCGACAGAGTCAGTTTTTGAATG
TTACAGCTACTGAAGATTATGTTGATCCTGTTACAAACCAGACGAAAACAAATGTGAAAGAGTTACAATA
CTACGTCGGACAGAGTTTTGCCAATGCAATGTACAATGCCTGCCGGGATGTGGAGGCCCCCTCAAGTAAT
GACAAGGCCCTGGGACTCCTGTGTGGGAAGGACGCTGACGCCTGTAATGCCACCAACTGGATTGAATACA
TGTTCAATAAGGACAATGGACAGGCACCTTTTACCATCACTCCTGTGTTTTCAGATTTTCCAGTCCATGG
GATGGAGCCCATGAACAATGCCACCAAAGGCTGTGACGAGTCTGTGGATGAGGTCACAGCACCATGTAGC
TGCCAAGACTGCTCTATTGTCTGTGGCCCCAAGCCCCAGCCCCCACCTCCTCCTGCTCCCTGGACGATCC
TTGGCTTGGACGCCATGTATGTCATCATGTGGATCACCTACATGGCGTTTTTGCTTGTGTTTTTTGGAGC
ATTTTTTGCAGTGTGGTGCTACAGAAAACGGTATTTTGTCTCCGAGTACACTCCCATCGATAGCAATATA
GCTTTTTCTGTTAATGCAAGTGACAAAGGAGAGGCGTCCTGCTGTGACCCTGTCAGCGCAGCATTTGAGG
GCTGCTTGAGGCGGCTGTTCACACGCTGGGGGTCTTTCTGCGTCCGAAACCCTGGCTGTGTCATTTTCTT
CTCGCTGGTCTTCATTACTGCGTGTTCGTCAGGCCTGGTGTTTGTCCGGGTCACAACCAATCCAGTTGAC
CTCTGGTCAGCCCCCAGCAGCCAGGCTCGCCTGGAAAAAGAGTACTTTGACCAGCACTTTGGGCCTTTCT
TCCGGACGGAGCAGCTCATCATCCGGGCCCCTCTCACTGACAAACACATTTACCAGCCATACCCTTCGGG
AGCTGATGTACCCTTTGGACCTCCGCTTGACATACAGATACTGCACCAGGTTCTTGACTTACAAATAGCC
ATCGAAAACATTACTGCCTCTTATGACAATGAGACTGTGACACTTCAAGACATCTGCTTGGCCCCTCTTT
CACCGTATAACACGAACTGCACCATTTTGAGTGTGTTAAATTACTTCCAGAACAGCCATTCCGTGCTGGA
CCACAAGAAAGGGGACGACTTCTTTGTGTATGCCGATTACCACACGCACTTTCTGTACTGCGTACGGGCT
CCTGCCTCTCTGAATGATACAAGTTTGCTCCATGACCCTTGTCTGGGTACGTTTGGTGGACCAGTGTTCC
CGTGGCTTGTGTTGGGAGGCTATGATGATCAAAACTACAATAACGCCACTGCCCTTGTGATTACCTTCCC
TGTCAATAATTACTATAATGATACAGAGAAGCTCCAGAGGGCCCAGGCCTGGGAAAAAGAGTTTATTAAT
TTTGTGAAAAACTACAAGAATCCCAATCTGACCATTTCCTTCACTGCTGAACGAAGTATTGAAGATGAAC
TAAATCGTGAAAGTGACAGTGATGTCTTCACCGTTGTAATTAGCTATGCCATCATGTTTCTATATATTTC
CCTAGCCTTGGGGCACATCAAAAGCTGTCGCAGGCTTCTGGTGGATTCGAAGGTCTCACTAGGCATCGCG
GGCATCTTGATCGTGCTGAGCTCGGTGGCTTGCTCCTTGGGTGTCTTCAGCTACATTGGGTTGCCCTTGA
CCCTCATTGTGATTGAAGTCATCCCGTTCCTGGTGCTGGCTGTTGGAGTGGACAACATCTTCATTCTGGT
GCAGGCCTACCAGAGAGATGAACGTCTTCAAGGGGAAACCCTGGATCAGCAGCTGGGCAGGGTCCTAGGA
GAAGTGGCTCCCAGTATGTTCCTGTCATCCTTTTCTGAGACTGTAGCATTTTTCTTAGGAGCATTGTCCG
TGATGCCAGCCGTGCACACCTTCTCTCTCTTTGCGGGATTGGCAGTCTTCATTGACTTTCTTCTGCAGAT
TACCTGTTTCGTGAGTCTCTTGGGGTTAGACATTAAACGTCAAGAGAAAAATCGGCTAGACATCTTTTGC
TGTGTCAGAGGTGCTGAAGATGGAACAAGCGTCCAGGCCTCAGAGAGCTGTTTGTTTCGCTTCTTCAAAA
ACTCCTATTCTCCACTTCTGCTAAAGGACTGGATGAGACCAATTGTGATAGCAATATTTGTGGGTGTTCT
GTCATTCAGCATCGCAGTCCTGAACAAAGTAGATATTGGATTGGATCAGTCTCTTTCGATGCCAGATGAC
TCCTACATGGTGGATTATTTCAAATCCATCAGTCAGTACCTGCATGCGGGTCCGCCTGTGTACTTTGTCC
TGGAGGAAGGGCACGACTACACTTCTTCCAAGGGGCAGAACATGGTGTGCGGCGGCATGGGCTGCAACAA
TGATTCCCTGGTGCAGCAGATATTTAACGCGGCGCAGCTGGACAACTATACCCGAATAGGCTTCGCCCCC
TCGTCCTGGATCGACGATTATTTCGACTGGGTGAAGCCACAGTCGTCTTGCTGTCGAGTGGACAATATCA
CTGACCAGTTCTGCAATGCTTCAGTGGTTGACCCTGCCTGCGTTCGCTGCAGGCCTCTGACTCCGGAAGG
CAAACAGAGGCCTCAGGGGGGAGACTTCATGAGATTCCTGCCCATGTTCCTTTCGGATAACCCTAACCCC
AAGTGTGGCAAAGGGGGACATGCTGCCTATAGTTCTGCAGTTAACATCCTCCTTGGCCATGGCACCAGGG
TCGGAGCCACGTACTTCATGACCTACCACACCGTGCTGCAGACCTCTGCTGACTTTATTGACGCTCTGAA
GAAAGCCCGACTTATAGCCAGTAATGTCACCGAAACCATGGGCATTAACGGCAGTGCCTACCGAGTATTT
CCTTACAGTGTGTTTTATGTCTTCTACGAACAGTACCTGACCATCATTGACGACACTATCTTCAACCTCG
GTGTGTCCCTGGGCGCGATATTTCTGGTGACCATGGTCCTCCTGGGCTGTGAGCTCTGGTCTGCAGTCAT
CATGTGTGCCACCATCGCCATGGTCTTGGTCAACATGTTTGGAGTTATGTGGCTCTGGGGCATCAGTCTG
AACGCTGTATCCTTGGTCAACCTGGTGATGAGCTGTGGCATCTCCGTGGAGTTCTGCAGCCACATAACCA
GAGCGTTCACGGTGAGCATGAAAGGCAGCCGCGTGGAGCGCGCGGAAGAGGCACTTGCCCACATGGGCAG
CTCCGTGTTCAGTGGAATCACACTTACAAAATTTGGAGGGATTGTGGTGTTGGCTTTTGCCAAATCTCAA
ATTTTCCAGATATTCTACTTCAGGATGTATTTGGCCATGGTCTTACTGGGAGCCACTCACGGATTAATAT
TTCTCCCTGTCTTACTCAGTTACATAGGGCCATCAGTAAATAAAGCCAAAAGTTGTGCCACTGAAGAGCG
ATACAAAGGAACAGAGCGCGAACGGCTTCTAAATTTCTAGCCCTCTCGCAGGGCATCCTGACTGAACTGT
GTCTAAGGGTCGGTCGGTTTACCACTGGACGGGTGCTGCATCGGCAAGGCCAAGTTGAACACCGGATGGT
GCCAACCATCGGTTGTTTGGCAGCAGCTTTGAACGTAGCGCCTGTGAACTCAGGAATGCACAGTTGACTT
GGGAAGCAGTATTACTAGATCTGGAGGCAACCACAGGACACTAAACTTCTCCCAGCCTCTTCAGGAAAGA
AACCTCATTCTTTGGCAAGCAGGAGGTGACACTAGATGGCTGTGAATGTGATCCGCTCACTGACACTCTG
TAAAGGCCAATCAATGCACTGTCTGTCCTCTCCTTTTTAGGAGTAAGCCATCCCACAAGTTCTATACCAT
ATTTTTAGTGACAGTTGAGGTTGTAGATACACTTTATAACATTTTATAGTTTAAAGAGCTTTATTAATGC
AATAAATTAACTTTGTACACATTTTTATATAAAAAAACAGCAAGTGATTTCAGAATGTTGTAGGCCTCAT
TAGAGCTTGGTCTCCAAAAATCTGTTTGAAAAAAGCAACATGTTCTTCACAGTGTTCCCCTAGAAAGGAA
GAGATTTAATTGCCAGTTAGATGTGGCATGAAATGAGGGACAAAGAAAGCATCTCGTAGGTGTGTCTACT
GGGTTTTAACTTATTTTTCTTTAATAAAATACATTGTTTTCCTAAAAAAAAAA
SEQ ID NO: 5-human NPC1 protein sequence-NCBI Reference Sequence
AAB63982.1
MTARGLALGLLLLLLCPAQVFSQSCVWYGECGIAYGDKRYNCEYSGPPKPLPKDGYDLVQELCPGFFFGN
VSLCCDVRQLQTLKDNLQLPLQFLSRCPSCFYNLLNLFCELTCSPRQSQFLNVTATEDYVDPVTNQTKTN
VKELQYYVGQSFANAMYNACRDVEAPSSNDKALGLLCGKDADACNATNWIEYMFNKDNGQAPFTITPVFS
DFPVHGMEPMNNATKGCDESVDEVTAPCSCQDCSIVCGPKPQPPPPPAPWTILGLDAMYVIMWITYMAFL
LVFFGAFFAVWCYRKRYFVSEYTPIDSNIAFSVNASDKGEASCCDPVSAAFEGCLRRLFTRWGSFCVRNP
GCVIFFSLVFITACSSGLVFVRVTTNPVDLWSAPSSQARLEKEYFDQHFGPFFRTEQLIIRAPLTDKHIY
QPYPSGADVPFGPPLDIQILHQVLDLQIAIENITASYDNETVTLQDICLAPLSPYNTNCTILSVLNYFQN
SHSVLDHKKGDDFFVYADYHTHFLYCVRAPASLNDTSLLHDPCLGTFGGPVFPWLVLGGYDDQNYNNATA
LVITFPVNNYYNDTEKLQRAQAWEKEFINFVKNYKNPNLTISFTAERSIEDELNRESDSDVFTVVISYAI
MFLYISLALGHIKSCRRLLVDSKVSLGIAGILIVLSSVACSLGVFSYIGLPLTLIVIEVIPFLVLAVGVD
NIFILVQAYQRDERLQGETLDQQLGRVLGEVAPSMFLSSFSETVAFFLGALSVMPAVHTFSLFAGLAVFI
DFLLQITCFVSLLGLDIKRQEKNRLDIFCCVRGAEDGTSVQASESCLFRFFKNSYSPLLLKDWMRPIVIA
IFVGVLSFSIAVLNKVDIGLDQSLSMPDDSYMVDYFKSISQYLHAGPPVYFVLEEGHDYTSSKGQNMVCG
GMGCNNDSLVQQIFNAAQLDNYTRIGFAPSSWIDDYFDWVKPQSSCCRVDNITDQFCNASVVDPACVRCR
PLTPEGKQRPQGGDFMRFLPMFLSDNPNPKCGKGGHAAYSSAVNILLGHGTRVGATYFMTYHTVLQTSAD
FIDALKKARLIASNVTETMGINGSAYRVFPYSVFYVFYEQYLTIIDDTIFNLGVSLGAIFLVTMVLLGCE
LWSAVIMCATIAMVLVNMFGVMWLWGISLNAVSLVNLVMSCGISVEFCSHITRAFTVSMKGSRVERAEEA
LAHMGSSVFSGITLTKFGGIVVLAFAKSQIFQIFYFRMYLAMVLLGATHGLIFLPVLLSYIGPSVNKAKS
CATEERYKGTERERLLNF

### Materials and Methods

### Construct Synthesis

AAV constructs encoding an eGFP and firefly luciferase reporter gene or therapeutic hNPC1 cDNA via 10 selected promoters were produced to evaluate activity and therapeutic efficacy. The 10 promoter sequences were designed at UCL and synthesised by GeneArt (ThermoFisher). The majority of the original pAAV.SYN.NPC1 construct was utilised to create these constructs, with the synthesised promoter sequences cloned into the pAAV.SYN.NPC1 construct via the Xhol and Kpnl restriction sites. Successful clones were confirmed by Sanger sequencing (Source Bioscience). A construct used in the study is shown in FIG. 17.

### In Vitro Transfection

Evaluation of functional transgene expression from newly produced constructs was undertaken by transfection into HEK293T cells. Cells were seeded into 24-well plates, with 50,000 cells per well grown in DMEM, 10% FBS, at 37° C. and 5% CO2. Seeded cells were transfected with 500 ng of the plasmid constructs (n=3 per group) 24 hours later, via linear polyethylenimine transfection (DNA:PEI ratio of 1:3). Cells were subsequently imaged 48 hours' post-transfection for eGFP expression or lysed with RIPA buffer (ThermoFisher) for protein purification.

### Western Blot

Levels of human NPC1 protein expression from the different constructs were evaluated by Western blot using an anti-NPC1 antibody (ab 134113, Abcam). Total protein concentration from cell and tissue lysates was normalised following a BCA protein assay (ThermoFisher). Lysates were mixed with LDS loading buffer (NuPage) and incubated at 70° C. for 20 minutes to denature proteins. 20-40 μg of total protein was loaded per well in a 4-12% Bis-Tris Mini gel (NuPage) and run at 180V for approximately 80 minutes. Proteins were subsequently transferred onto PDVF (Merck Millipore) membrane via semi-dry transfer, blocked with 5% bovine serum albumin (BSA, Sigma) in lx tris-buffered saline with 0.1% Tween-20 (TBS-T) at room temperature for 30 minutes and incubated with primary antibody in TBST-T with 3% BSA overnight at 4° C. Membranes were then washed, incubated with HRP conjugated secondary antibodies and visualised via chemiluminescence.

### Primary Culture Transduction

To evaluate transgene expression in different cells of the nervous system, primary cultures from the brains of E18 wildtype CD1 embryos were conducted. Whole brains were extracted from E18 embryos, disassociated with trypsin and 200,000 cells were seeded per well on to laminin and polylysine coated coverslips in 6-well plates. 48 hours post-seeding, cells were transduced with selected AAV9 vectors at a multiplicity of infection of 150,000 viral genomes per cell. 5-days post-transduction the cells were washed, fixed with 4% paraformaldehyde and underwent immunofluorescent staining.

### In Vivo Administration

To evaluate transgene expression from the different constructs in vivo, Npc1^{-/-} and Npc1^{nmf164} mice were administered at birth with AAV9-hNPC1 via intracerebroventricular (ICV) injection with a 33-gauge Hamilton syringe. A total dose of 5E10 vg was administered in a volume of 10 μL, with 5 μL injected into each hemisphere. Pups were then returned to the dam to recover. Alternatively, wildtype mice were administered with AAV9-NLSeGFP.2A.FLuc for reporter gene studies.

### Reporter Gene Imaging

Mice injected at birth with 5E10 vg of AAV9-NLSeGFP.2A.FLuc regulated by the selected promotors underwent bioluminescent imaging (IVIS Lumina, PerkinElmer) 50 days post-administration to evaluate levels and distribution of luciferase transgene expression in different organs. Prior to imaging, mice were injected with D-luciferin at a dose of 150 mg/kg, left for 10 minutes and dissected. Bioluminescence imaging was subsequently carried out with a binning factor of 4, a 1.2/f stop and open filter. Regions of interest were defined manually around each organ under investigation. Signal intensities were calculated using Living Image software (PerkinElmer) and expressed as photons per second per cm² per steradian (Radiance).

### In Vivo Monitoring

The weights of Npc1^{-/-} and Npc1^{nmf164} mice administered with AAV9-hNPC1 and control groups were monitored weekly, with a humane endpoint of 15% total weight loss. Behavioural assessments were carried out on Npc1^{-/-} mice at 10 weeks of age to assess therapeutic efficacy of the different vectors.

Automated gait analysis was performed using the CatWalk system (Noldus), where mice were filmed walking a minimum of 3 times across a backlit stage. Runs were assigned and analysed using the CatWalk XT software v9.1 (Noldus) to produce footprint, stride and overall run measurements. Parameters measured include regularity index (% index for the degree of inter-limb coordination during gait), swing speed (speed of the paw between successive paw placement), stride length (the distance between successive paw placement of the same paw) and duration (run duration).

Tremor was measured using a commercial tremor monitor (San Diego Instruments), according to the manufacturer's instructions. Mice were placed inside the apparatus on an anti-vibration table and monitored for 256 s, after 30 s of acclimatization time. The output was subsequently analysed for high frequency tremor (32-55 Hz) and presented as average tremor intensity (dBV).

### Tissue Analysis

Following terminal perfusion with PBS, organs were dissected, fixed for 48 hours with 4% paraformaldehyde in PBS and transferred into 30% sucrose in PBS for cryoprotection. Tissue was then cryosectioned at -20° C. using a cryostat microtome to 20 μm thickness. Immunohistochemical staining was used to evaluate transgene expression levels and pathological markers. Endogenous peroxidase activity was depleted by incubating sections in 1% H₂O₂ in TBS for 30 min and washed 3 times in TBS, after which endogenous non-specific protein binding was blocked by incubation in 15% normal serum (Sigma) in TBS-T (TBS with 0.3% Triton X-100) for 30 min. Sections were incubated overnight in 10% normal serum in TBS-T with primary antibodies for NPC1 (1:500, ab134113, Abcam), GFAP (1:2000, MAB3402, Merck Millipore), CD68 (1:2000, MCA1957, AbD Serotech) or Calbindin (1:10 000, CB38, Swant). Following washes in TBS, sections were incubated in 10% normal serum in TBS-T with biotinylated secondary antibodies anti-rabbit, anti-rat or anti-mouse IGg (1:1000, Vector Laboratories) for 2 hours. Staining was visualized using Vectastain avidin-biotin solution (ABC, Vector Laboratories) and DAB (Sigma), after which the sections were mounted, dehydrated, cleared in histoclear (National Diagnostics) for 30 min and finally coverslipped with DPX (VWR). Representative images were captured using a live video camera (Zeiss) mounted onto a Zeiss Axiolab light microscope.

### Example 1: Evaluation of Reporter Gene Expression from Selected Promoters

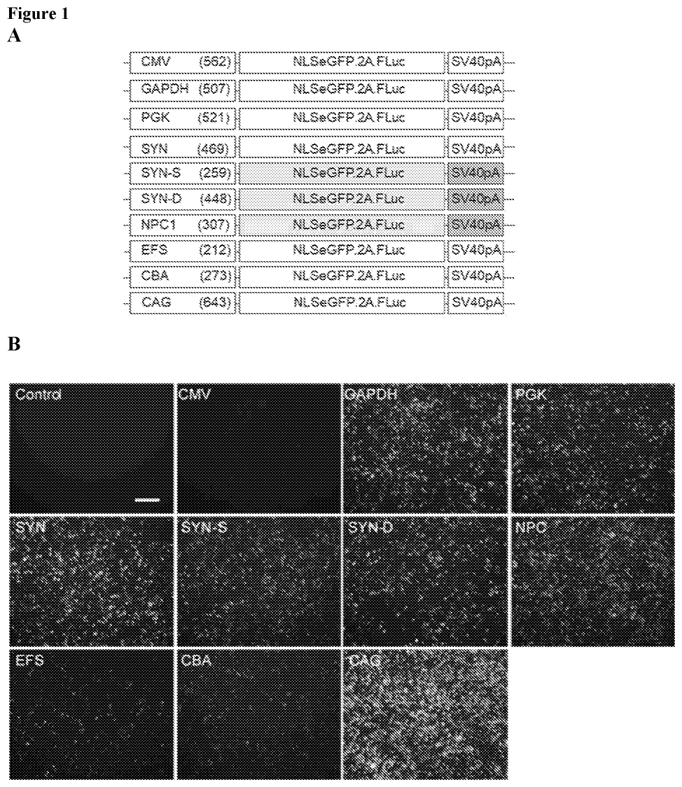

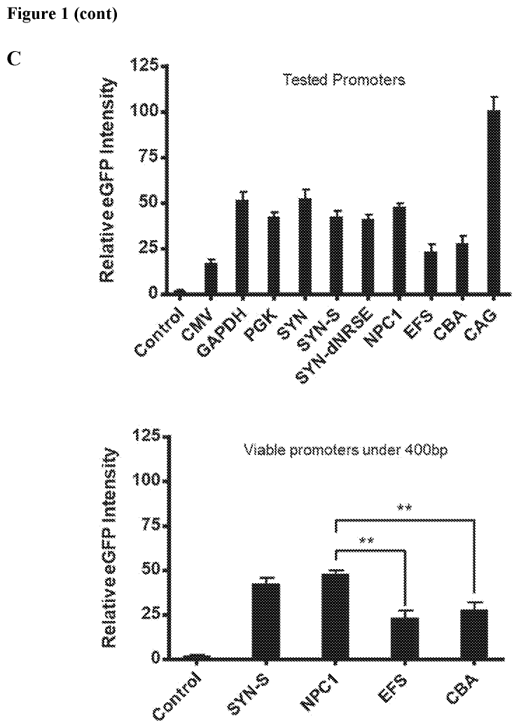

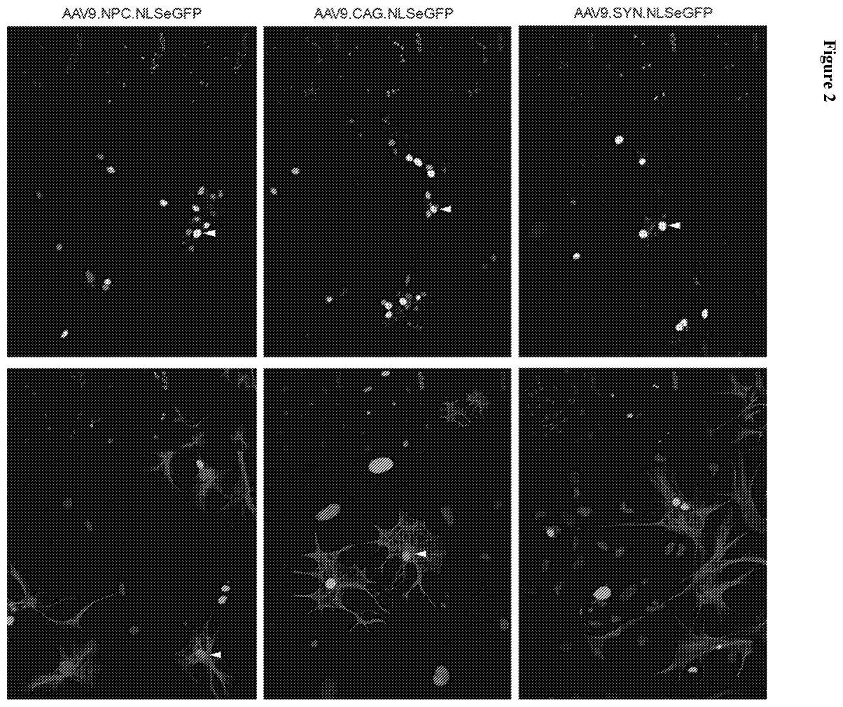

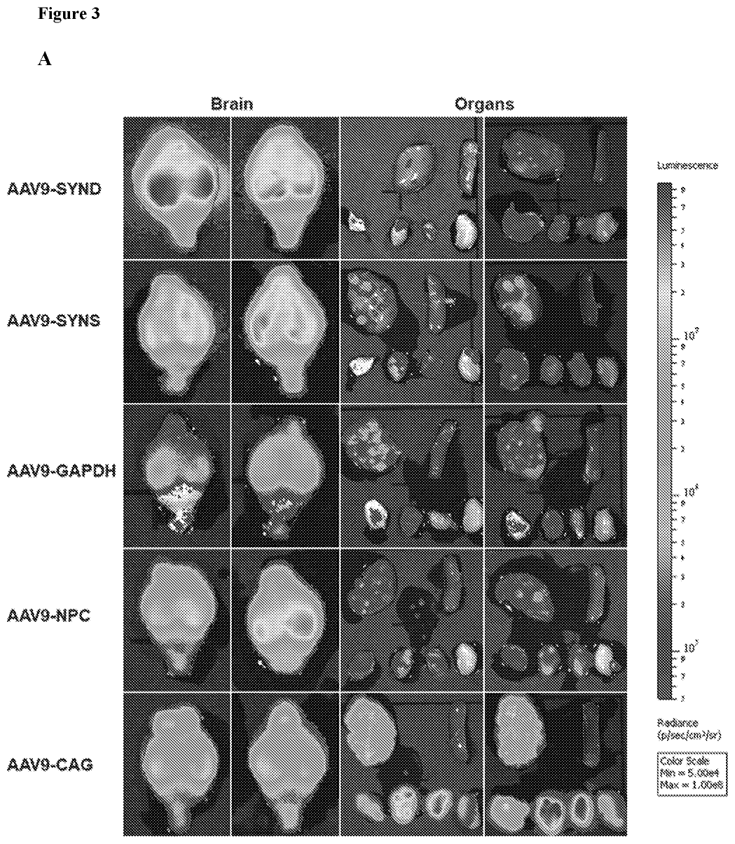

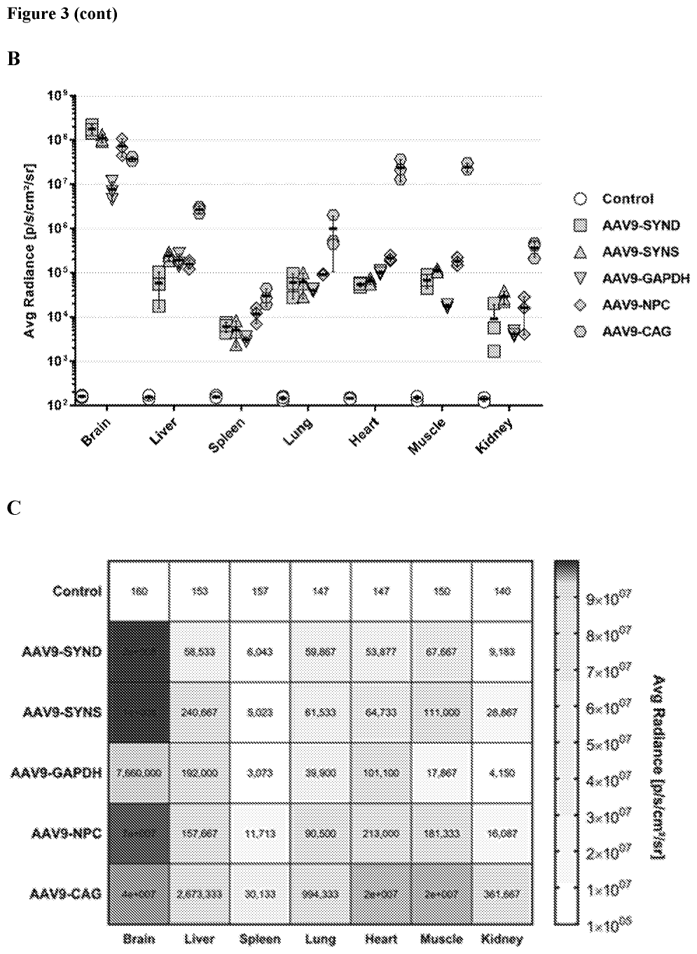

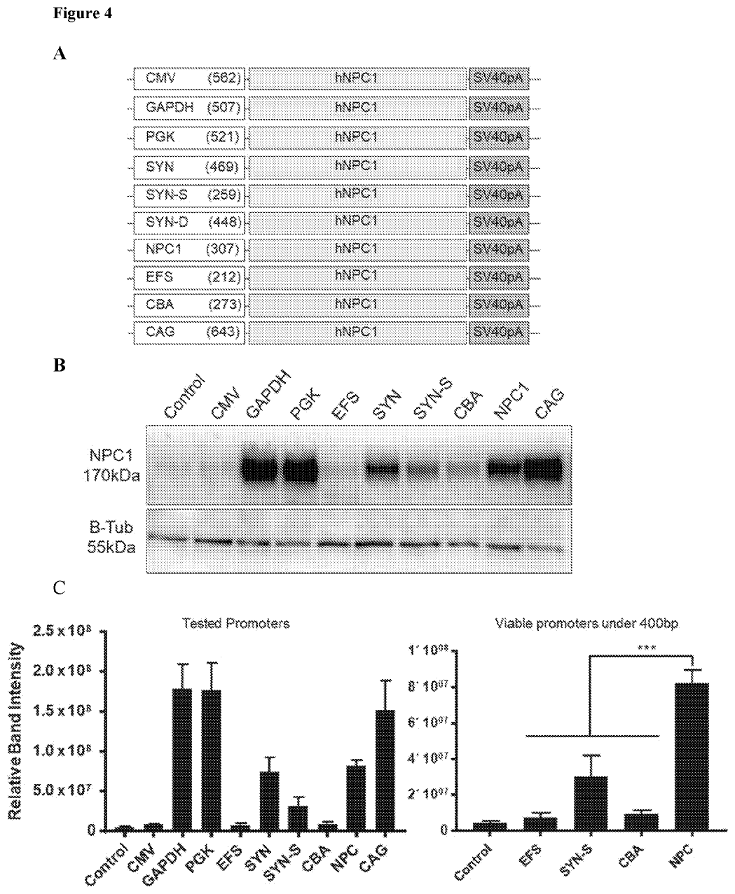

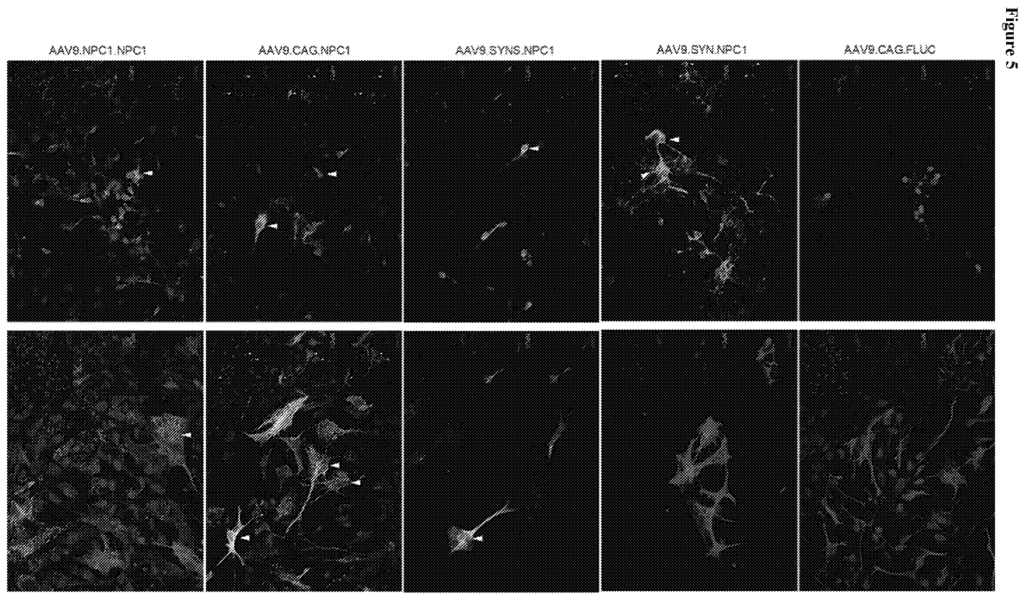

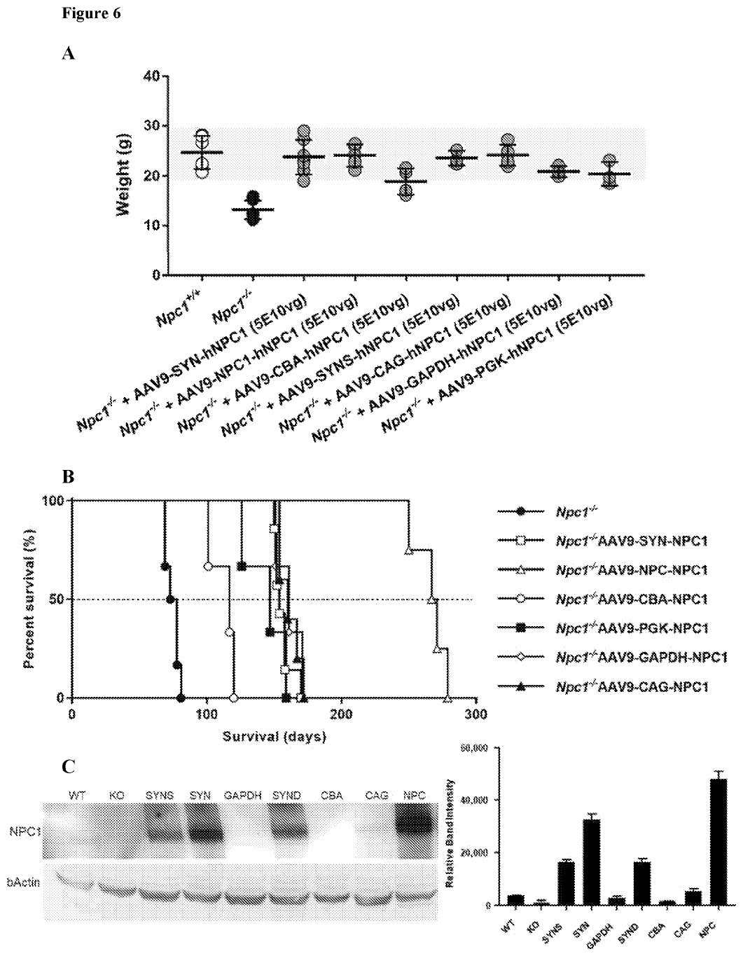

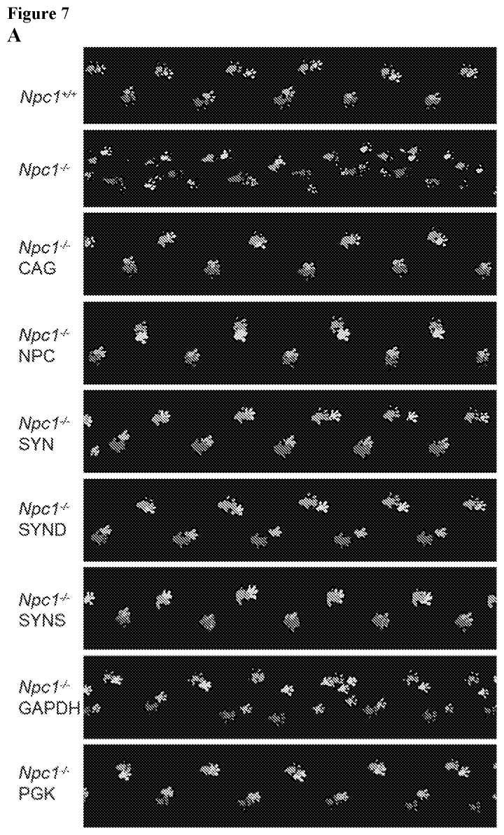

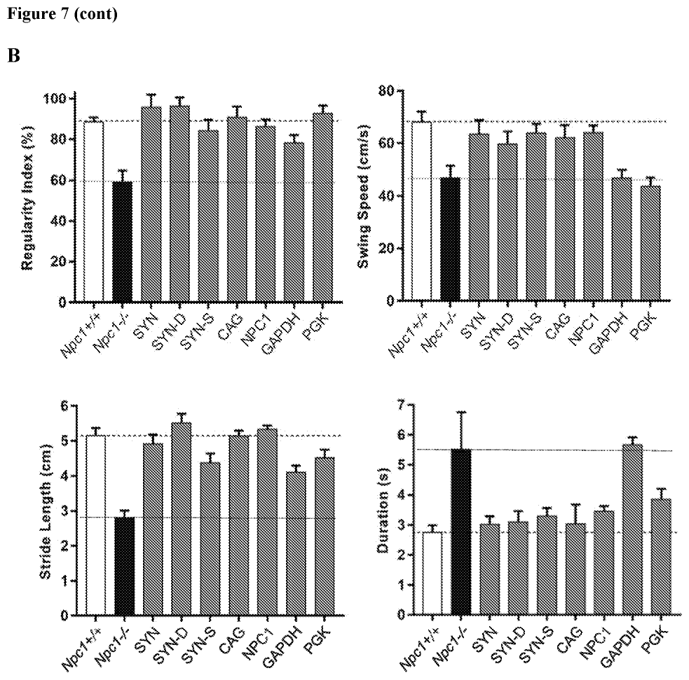

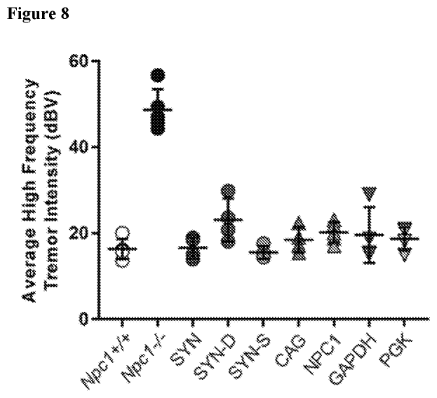

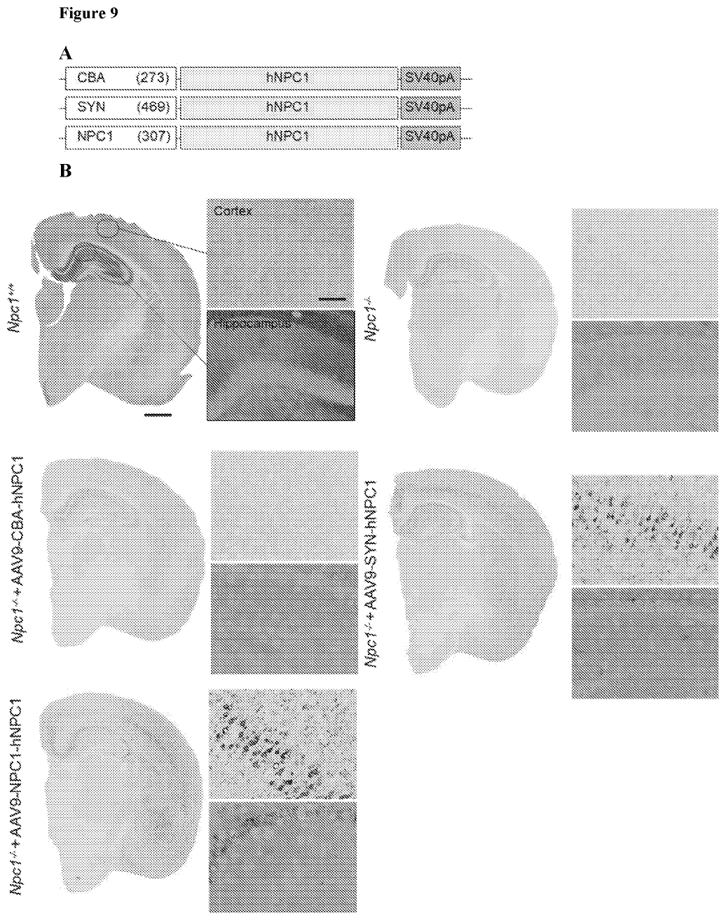

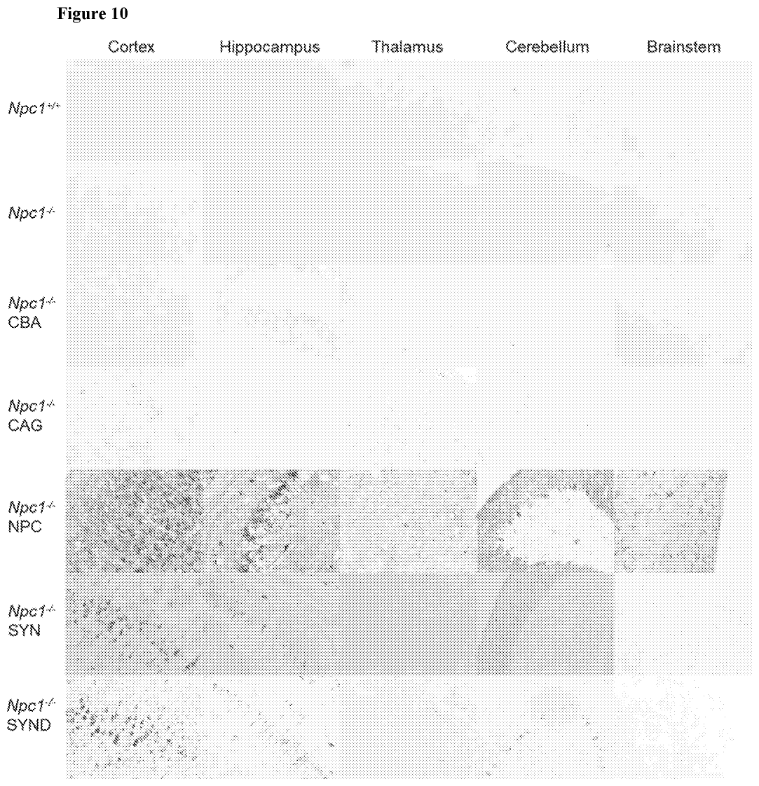

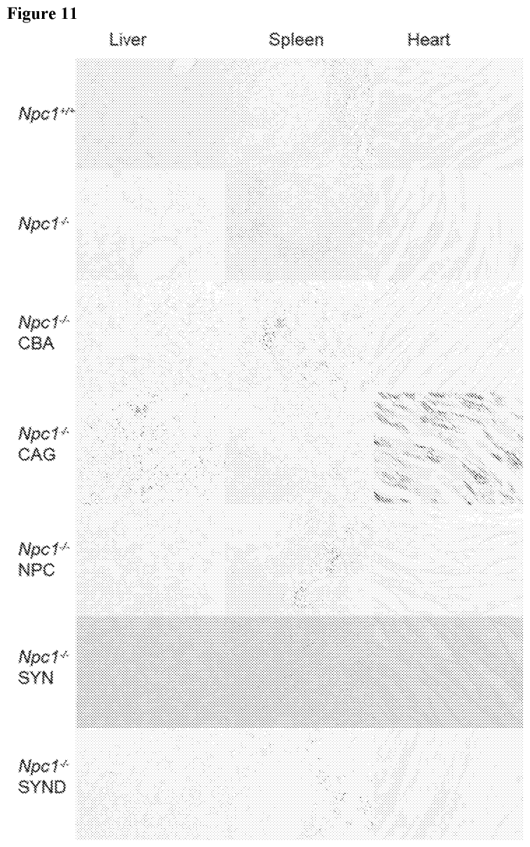

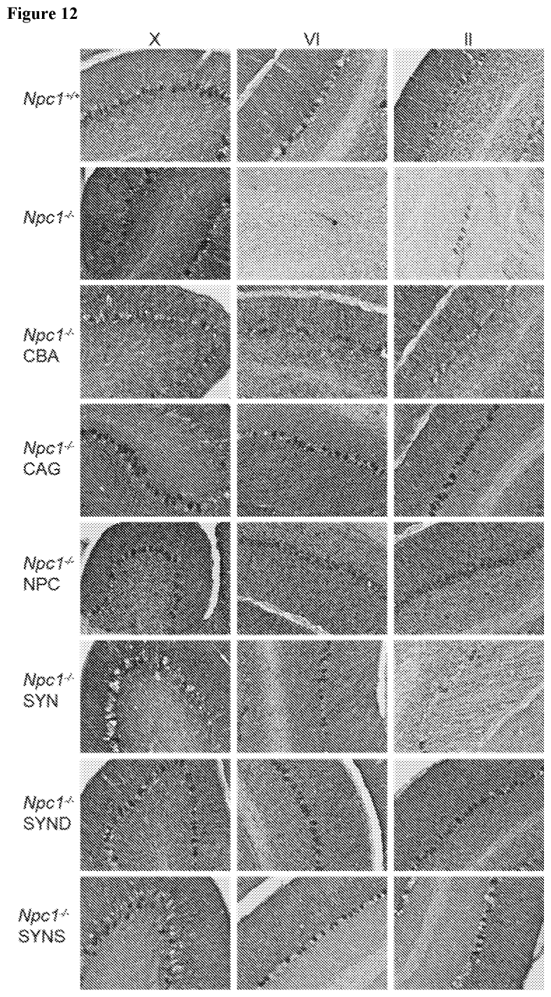

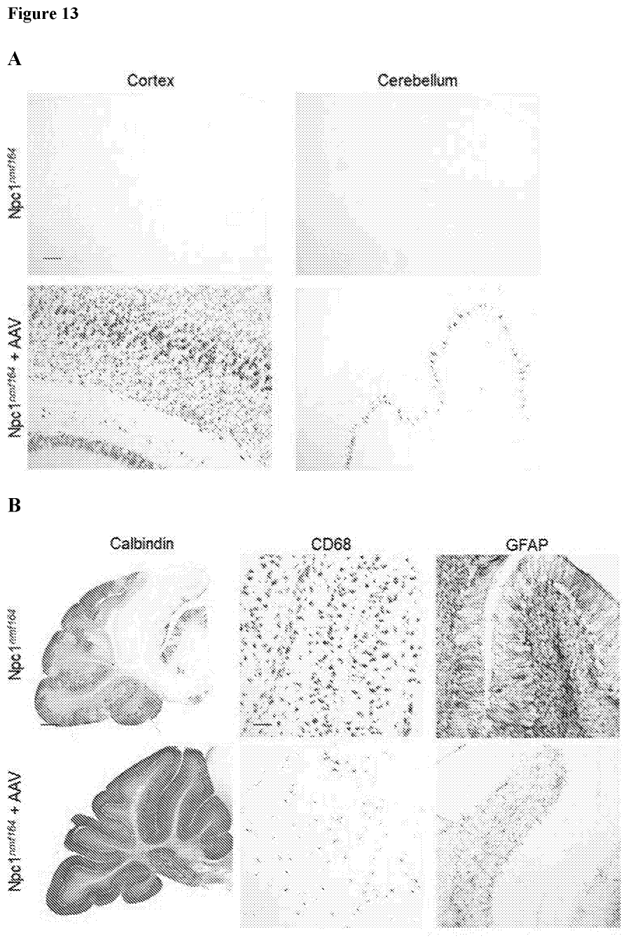

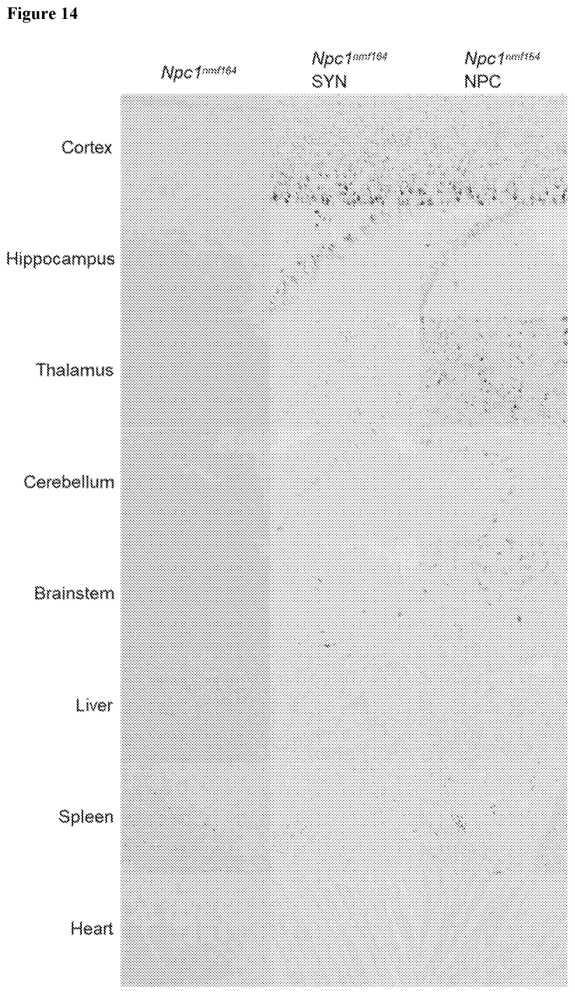

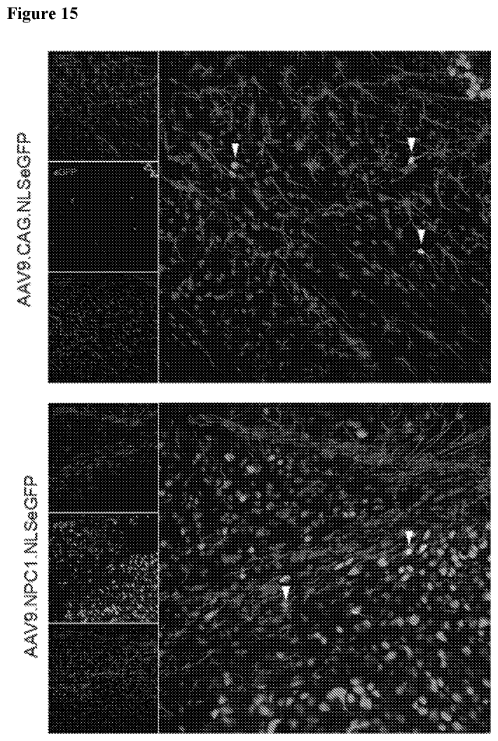

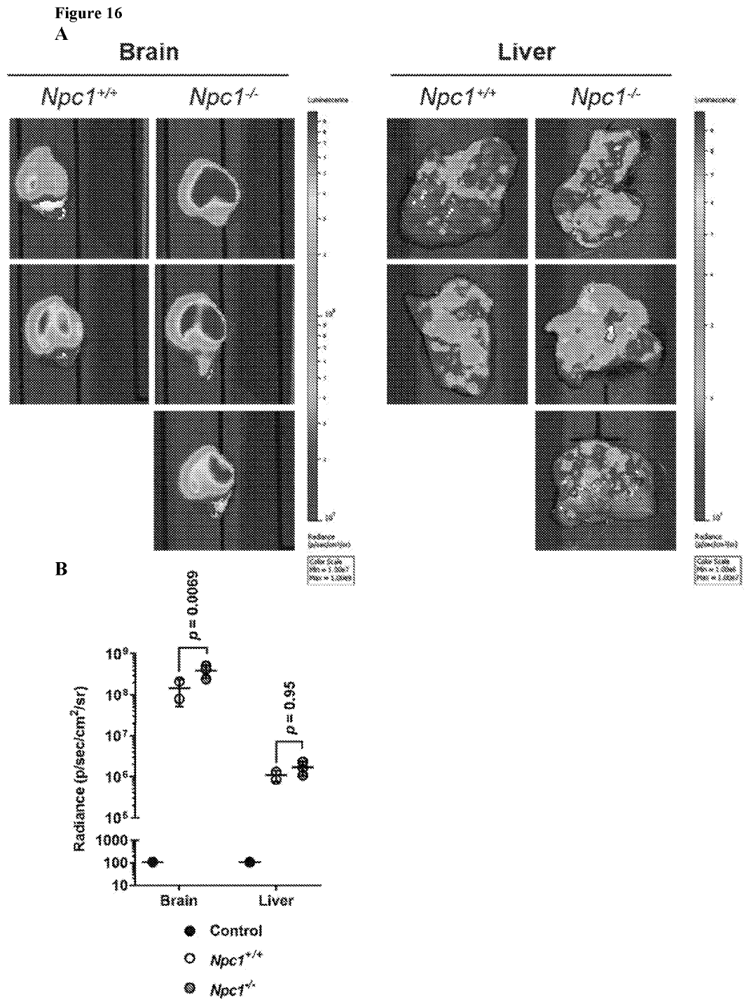

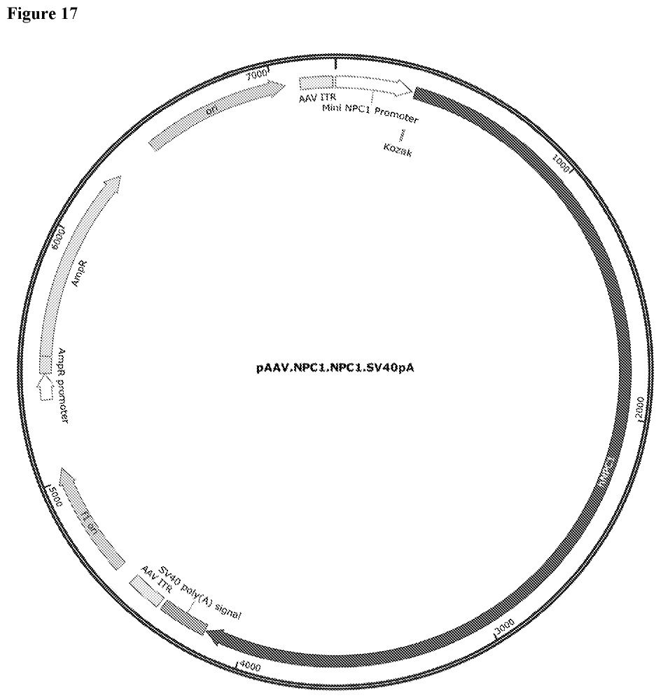

Selected promoter sequences were cloned into the pAAV.NLSeGFP.2A.FLuc plasmid to create 10 reporter constructs (FIG. 1A). To confirm functionality and evaluate initial eGFP reporter gene expression levels, HEK293T cells were transfected with each construct (FIG. 1B). Under UV imaging, positive eGFP expression was observed with each construct, confirming that the promoter sequences and constructs were functional. To get a first indication on promoter strength in vitro, 5 non-overlapping images of each well (n=3 wells per group) were taken and analysed with Image-Pro Premier software (Media Cybernetics), measuring relative eGFP intensity compared to background auto-fluorescence (FIG. 1C). On average, non-transfected control cells showed no eGFP expression, whereas the positive control promoter sequence CAG demonstrated the highest levels of eGFP protein. Of the viable promoters under 400 bp most suitable for working with hNPC1, the shortened synapsin (SYN-S) and endogenous NPC1 promoter (NPC1) fragments performed the best, with expression levels comparable to the synapsin promoter (SYN) used in our initial studies.

These validated constructs were subsequently used for viral vector purification to produce AAV9-NLSeGFP.2A.FLuc reporter vectors containing the selected promoter sequences. To validate the functionality of these reporter vectors and confirm their ability to express in both neuronal and glial cells, primary brain cultures from E18 wildtype embryos were transduced and stained for nuclear localised eGFP and cellular markers (FIG. 2). As expected from our previous studies, the neuronal selective SYN promoter demonstrated strong positive eGFP expression in NeuN positive neuronal cells, but no eGFP was observed in GFAP positive astrocytes. In comparison, the strong ubiquitous positive control promoter CAG demonstrated eGFP expression in both NeuN and GFAP positive cells. Similarly, the endogenous NPC1 promoter sequence also demonstrated eGFP expression in both neurons and astrocytes, confirming its ability to express in different cell types.

The AAV9-NLSeGFP.2A.FLuc vectors containing the different promoters were subsequently injected ICV into newborn wildtype mice (n=3 per group) to evaluate in vivo luciferase reporter gene expression levels in different organs (FIG. 3A). Bioluminescence was observed in the brains and organs of all ICV AAV9-NLSeGFP.2A.FLuc injected mice, as opposed to PBS injected controls. High levels of luciferase expression were observed in the brains with SYND, SYNS, NPC and CAG promoters, compared to low levels with the GAPDH promoter (FIG. 3B/C). As expected the SYND and SYNS promoters (variations of the original SYN promoter) demonstrated the highest levels of reporter gene expression in the brain with relatively low levels in visceral organs. In comparison, the strong ubiquitous CAG promoter showed lower levels of activity in the brain but very high activity in the visceral organs.

Interestingly, the endogenous NPC1 promoter fragment exhibited high levels of activity in both the brain and visceral organs, indicating its ubiquitous nature. Although NPC1 promoter activity wasn't as high in the visceral organs as with the positive control CAG promoter it remained superior in the brain.

### Example 2: In Vitro Evaluation of hNPC1 Gene Expression from Selected Promoters

Building on the positive results from the reporter gene studies, constructs were produced containing the wildtype hNPC1 cDNA being expressed by the 10 selected promoter sequences (FIG. 4A). To confirm functionality and evaluate initial hNPC1 protein expression levels in vitro, HEK293T cells were transfected with each construct (n=3 wells per group) and resulting protein lysates were analysed by Western blot for hNPC1 (FIG. 4B). Quantification of the hNPC1 band intensity was normalised to loading control β-tubulin levels (FIG. 4C). Similar to the reporter gene studies, high levels of hNPC1 protein was observed with the positive control CAG promoter, along with the other relatively long GAPDH and PGK promoters. Again, between the short promoter sequences the NPC1 promoter fragment performed the best, with the hNPC1 levels comparable to the longer SYN promoter used in our previous studies.

These validated constructs were subsequently used for viral vector purification to produce AAV9-hNPC1 vectors containing the selected promoter sequences. To validate the functionality of these vectors and confirm their ability to express in both neuronal and glial cells, primary brain cultures from E18 wildtype embryos were transduced and stained for hNPC1 and cellular markers (FIG. 5). Minimal endogenous murine NPC1 protein was observed in the negative control following transduction with a AAV9-CAG-FLuc reporter gene vector. As expected from our previous studies, the neuronal selective SYN promoter demonstrated strong positive hNPC1 expression in NeuN positive neuronal cells, yet minimal endogenous levels in GFAP positive astrocytes. In comparison, the strong ubiquitous positive control promoter CAG demonstrated hNPC1 expression in both NeuN and GFAP positive cells.

Similar to the CAG promoter and previous reporter gene study, the endogenous NPC1 promoter sequence also demonstrated hNPC1 expression in both neurons and astrocytes, confirming its ability to express hNPC1 in different cell types.

### Example 3: In Vivo Evaluation of AAV9-hNPC1 Therapeutic Efficacy from Selected Promoters

These vectors were subsequently evaluated for their therapeutic efficacy in the well characterised Npc1^{-/-} mouse model. Npc1^{-/-} mice were injected ICV with 5E10 vg at birth and monitored weekly (n=6). The first indication of therapeutic efficacy was measured on average body weight at 10 weeks of age (FIG. 6A). All AAV9-hNPC1 vectors with the selected promoters increased week 10 body weight from untreated levels back to within the wildtype range, except for Npc1^{-/-} mice treated with the CBA vector, where the average weight was significantly below average wildtype weights.

To evaluate survival rate, treated Npc1^{-/-} mice were kept alive until they reached the humane point of 15% loss of total body weight (FIG. 6B). As our previous studies have demonstrated, untreated Npc1^{-/-} mice have a lifespan on average of 70 days. All Npc1^{-/-} mice treated with AAV9-hNPC1 containing the different promoters survived beyond their expected lifespan. Following on from the low levels of hNPC1 protein expression seen in the in vitro study, Npc1^{-/-} mice treated with the CBA AAV9-hNPC1 vector demonstrated the lowest increase in lifespan, reaching on average 112 days. Despite high levels of reporter gene expression and hNPC1 expression in the previous in vitro studies, Npc1^{-/-} mice treated with the CAG (161 days), GAPDH (160 days) and PGK (144 days) vectors demonstrated survival comparable to our original SYN (155 days) studies. Surprisingly, Npc1^{-/-} mice treated with the NPC1 promoter vector demonstrated a very significant extension in lifespan compared to all other evaluated promoters, reaching an average lifespan of 265 days.

Western blot analysis of brain lysates from treated Npc1^{-/-} mice for hNPC1 protein expression (FIG. 6C), revealed low levels of NPC1 protein in untreated wildtype mice and Npc1^{-/-} mice treated with GAPDH and CBA AAV9-hNPC1 vectors. As expected, untreated Npc1^{-/-} mice showed no NPC1 protein. Notably, despite the high levels of hNPC1 protein expression measured in initial in vitro studies, Npc1^{-/-} mice treated with the positive control CAG vector only achieved wildtype NPC1 levels. This may be related to the oversized viral genome that results from incorporating the relatively large CAG promoter together with the large hNPC1 cDNA. In comparison, shortened versions of the synapsin promoter (SYNS and SYND) achieved higher levels of hNPC1 expression, compared to the original SYN promoter. Surprisingly, despite the small promoter size, Npc1^{-/-} mice treated with the NPC1 promoter AAV-hNPC1 vector demonstrated a larger than 10-fold increase in NPC1 protein levels compared to wildtype mice and a 1.5-fold increase compared to SYN treated Npc1^{-/-} mice in the brain.

Gait and tremor analysis (n=3 per group) was additionally performed at the 10-week time point to further assess therapeutic efficacy from the selected promoters. Automated gait analysis revealed the normalisation of gait back to wildtype form in Npc1^{-/-} mice treated with most of the selected promoters, compared to untreated Npc1^{-/-} mice (FIG. 7A/B). Similarly, high frequency tremor analysis conducted at 10 weeks of age also showed correction of the phenotype back to wildtype levels in Npc1^{-/-} mice treated with the different promoters, compared to untreated Npc1^{-/-} mice (FIG. 8).

To evaluate distribution of hNPC1 protein expression from the selected promoters following

ICV administration of AAV9-hNPC1, cryosectioned brain (FIGS. 9, 10) and visceral organ tissue (FIG. 11) underwent anti-NPC1 staining. As previously published, the anti-NPC1 antibody is not ideal as only faint staining of endogenous murine NPC1 was visible. Untreated Npc1^{-/-} mice demonstrate a complete absence of NPC1 staining. In the brain of Npc1^{-/-} mice, minimal NPC1 staining was observed following treatment with the CBA promoter. Correlating to the low NPC1 levels seen via Western blot, Npc1^{-/-} mice treated with the CAG promoter revealed relatively low levels of NPC1 protein, similar to wildtype levels. In comparison, Npc1^{-/-} mice treated with SYN or SYN-S demonstrated strong NPC1 staining in several brain regions. However, surprisingly the highest levels of NPC1 staining was achieved with the NPC1 promoter with positive expression seen throughout all monitored brain regions.

Purkinje neuron loss in the cerebellum is one of the hallmarks of NP-C disease, which is mirrored in the Npc1^{-/-} mouse model and can be visualised by calbindin staining (FIG. 12). Npc1^{-/-} mice treated with the CBA promoter demonstrated limited rescue of Purkinje neurons, correlating with the low levels of NPC1 expression previously observed. In comparison, CAG, NPC and SYNS treated Npc1^{-/-} mice showed calbindin staining comparable to wildtype mice, indicating significant rescue of Purkinje neuron loss.

Although the Npc1^{-/-} mouse model mirrors certain aspects of human NP-C disease, Npc1^{-/-} mice have the most of their Npc 1 gene deleted and therefore do not produce any NPC1 protein. However, the majority of patients have NP-C due to missense mutations. These patients produce a certain amount of NPC1 protein that is non-functional or rapidly degraded. The inventors therefore decided to evaluate our therapy in a second murine model of NP-C. Npc1^{nmf164} mice produce low levels of non-functional murine NPC1 protein and disease progression is slower than in the Npc1^{-/-} mouse model, mirroring the average NP-C patient more accurately. Npc1nmf¹⁶⁴ mice were administered ICV at birth with 5E10 vg of AAV9-hNPC1 with either the original synapsin (SYN) promoter or our new NPC1 promoter fragment. At their humane endpoint of 14 weeks, administered Npc1^{nmf164} mice were culled and tissue was processed for immunohistochemistry. Similar to our previous studies with the Npc1^{-/-} mice, Npc1nmf¹⁶⁴ mice administered with AAV9-hNPC1 containing the NPC1 promoter sequence showed high levels of human NPC1 protein throughout all monitored brain regions, when compared to untreated Npc1^{nmf164} mice, where minimal staining was observed (FIG. 13). Compared to the SYN promoter, NPC1 protein expression was also more widespread throughout the brain with the NPC1 promoter (FIG. 14). Calbindin staining revealed significant Purkinje neuron loss in untreated Npc1^{nmf164} mice which was rescued by neonatal treatment with AAV9-hNPC1. The inventors additionally observed significant improvement in neuroinflammation via CD68 (microglial) and GFAP (astrocytic) staining (FIG. 13B) within the cerebellum of Npc1^{nmf64} mice treated with AAV9-hNPC1 containing the NPC promoter, which the inventors didn't observe with their previous SYN promoter. FIG. 15 demonstrates confirmation of eGFP reporter gene expression in astrocytes with the NPC1 promoter, which was not previously seen with the synapsin promoter.

Finally to evaluate if the high levels of NPC1 promoter activity the inventors observed in certain experiments were linked to the genotype of the mouse, the inventors administered their previously produced reporter vector AAV9-NLSeGFP.2A.FLuc containing the NPC1 promoter ICV into newborn wildtype and homozygous Npc1^{-/-} mice. The brains and livers of injected mice were imaged 10 weeks later revealing high levels luciferase expression in both organs (FIG. 16A). Interestingly, the brains from homozygous Npc1^{-/-} mice demonstrated elevated levels of bioluminescence compared to wildtype mice administered with the same viral vector dose (FIG. 16B). This effect however was not mirrored in the liver. This experiment indicates that NPC1 promoter activity may be higher in an environment of NP-C pathology.

### REFERENCES

Altschul S. F. A protein alignment scoring system sensitive at all evolutionary distances J. Mol. Evol. 36, 290-300 (1993).
Altschul, S. F. et al., Basic local alignment search tool J. Mol. Biol., 215, 403-410 (1990).
Coura Rdos S. and Nardi, N. B., The state of the art of adeno-associated virus-based vectors in gene therapy, Virol. J., 4, 99 (2007).
Chandler R. J., et al., (2017) Systemic AAV9 gene therapy improves the lifespan of mice with Niemann-Pick disease, type C1. Hum. Mol. Genet., 26, 52-64.
Choi et al. (2005) AAV hybrid serotypes: improved vectors for gene delivery. Curr Gene Ther. 5, 299-310.
Devereux, J. et al. A comprehensive set of sequence analysis programmes for the VAX. Nucl. Acids Res. 12, 387-395 (1984).
Dong, J. Y. et al., Quantitative analysis of the packaging capacity of recombinant adeno-associated virus, Hum. Gene Ther., 7, 2101-2112 (1996).
Erles, K., Sebokova, P. and Schlehofer, J. R., Update on the prevalence of serum antibodies (IgG and IgM) to adeno-associated virus (AAV), J Med. Virol. 59, 406-411 (1999).
Henikoff, S. and Henikoff, J. G., Proc. Natl. Acad. Sci. USA 89, 10915-10919 (1992).
Hermonat, P. L. et al., Genetics of adeno-associated virus: isolation and preliminary characterization of adeno-associated virus type 2 mutants, J. Virol., 51, 329-339 (1984).
Higgins, M.E. et al., (1999). Niemann-Pick C1 is a late endosome-resident protein that transiently associates with lysosomes and the trans-Golgi network. Molecular genetics and metabolism, 68,1-13.
Hughes, M P et al., (2018), AAV9 intracerebroventricular gene therapy improves lifespan, locomotor function and pathology in a mouse model of Niemann—Pick type C1 disease, Human Molecular Genetics, 27, 3079-3098.
Karlin, S. and Altschul, S. F., (1993) Applications and statistics for multiple high-scoring segments in molecular sequences Proc. Natl. Acad. Sci. USA 90: 5873-5787.
Kotin, R. M. et al., (1990) Site-specific integration by adeno-associated virus, Proc. Natl. Acad. Sci U.S.A 87, 2211-2215.
Lloyd-Evans E., et al., (2008) Niemann-Pick disease type C1 is a sphingosine storage disease that causes deregulation of lysosomal calcium. Nat. Med., 14, 1247-1255.
Lloyd-Evans E. and Platt F. M. (2010) Lipids on trial: the search for the offending metabolite in Niemann-Pick type C disease. Traffic, 11, 419-428.
Manno, C. S. et al., Successful transduction of liver in hemophilia by AAV-Factor IX and limitations imposed by the host immune response, Nat. Med. (2006).
Michelfelder, S. and Trepel, M., Adeno-associated viral vectors and their redirection to cell-type specific receptors, Adv. Genet., 67, 29-60 (2009).
Moskalenko, M. et al., Epitope mapping of human anti-adeno-associated virus type 2 neutralizing antibodies: implications for gene therapy and virus structure, J Virol., 74, 1761-1766 (2000).
Summerford, C. and Samulski, R. J., Membrane-associated heparan sulfate proteoglycan is a receptor for adeno-associated virus type 2 virions, J. Virol., 72, 1438-1445 (1998).
Sun, J. Y. et al., Immune responses to adeno-associated virus and its recombinant vectors, 10, 964-976 (2003).
Te Vruchte, D. et al., (2004). Accumulation of glycosphingolipids inNiemann-Pick C disease disrupts endosomal transport. Journal of Biological Chemistry, 279(25), pp.26167-26175.
Thomas, C. E. et al., Rapid uncoating of vector genomes is the key to efficient liver transduction with pseudotyped adeno-associated virus vectors, J. Virol. 78, 3110-3122 (2004).
Vanier, M. T., 2010. Niemann-Pick disease typeC. Orphanet journal of rarediseases, 5, p.16.
Vanier, M. T. et al., 1996. Genetic heterogeneity in Niemann-Pick C disease: a study using somatic cell hybridization and linkage analysis. American journal of humangenetics, 58(1), pp. 118-125.
Vincent-Lacaze, N. et al., Structure of adeno-associated virus vector DNA following transduction of the skeletal muscle, J Virol. 73, 1949-1955 (1999).
Wu, Z. et al., Adeno-associated virus serotypes: vector toolkit for human gene therapy, Mol. Ther., 14, 316-327 (2006).
Xie C., et al., (2017) AAV9-NPC1 significantly ameliorates Purkinje cell death and behavioral abnormalities in mouse NPC disease. J. Lipid Res., 58, 512-518.
Zaiss, A. K. and Muruve, D. A., Immunity to adeno-associated virus vectors in animals and humans: a continued challenge. Gene Ther 15, 808-816 (2008).

1. An expression construct comprising in a 5′ to 3′ direction:
(a) a NPC1 promoter fragment nucleotide sequence consisting of no more than 400 nucleotides in length, wherein the sequence comprises at least 250 consecutive nucleotides from SEQ ID NO: 1, or a sequence having at least 90% sequence identity to said promoter fragment sequence that retains the functionality of the NPC1 promoter; and
(b) (i) the hNPC1 nucleotide sequence as shown in SEQ ID NO: 2 or 4, or a sequence having at least 70% sequence identity to SEQ ID NO: 2 or 4 that retains the functionality of hNPC1; or (ii) a hNPC1 nucleotide sequence encoding the polypeptide as shown in SEQ ID NO: 3 or 5, or a polypeptide having at least 90% sequence identity to the polypeptide of SEQ ID NO: 3 or 5 that retains the functionality of hNPC1.

2. The expression construct of claim 1, wherein the NPC1 promoter fragment sequence comprises least 280 consecutive nucleotides from SEQ ID NO: 1, or a sequence having at least 90% sequence identity to said promoter fragment sequence that retains the ability to express hNPC1.

3. The expression construct of claim 2, wherein the NPC1 promoter fragment sequence consists of no more than 350 nucleotides in length and comprises at least 290 consecutive nucleotides from SEQ ID NO: 1, or a sequence having at least 90% sequence identity to said promoter fragment sequence that retains the ability to express hNPC1.

4. The expression construct of claim 3, wherein the NPC1 promoter fragment sequence consists of SEQ ID NO: 1.

5. The expression construct of any one of the preceding claims, wherein the sequence of (b)(i) has at least 80% sequence identity to SEQ ID NO: 2 or 4.

6. The expression construct of claim 5, wherein the sequence of (b)(i) has at least 90% sequence identity to SEQ ID NO: 2 or 4.

7. The expression construct of any one of claims 1 to 4, wherein the sequence of (b)(ii) has at least 95% sequence identity to SEQ ID NO: 3 or 5.

8. The expression construct of any one of claims 1 to 4, wherein the
(a) hNPC1 nucleotide sequence encoding the polypeptide of SEQ ID NO: 3 is SEQ ID NO: 2; or
(b) hNPC1 nucleotide sequence encoding the polypeptide of SEQ ID NO: 5 is SEQ ID NO: 4.

9. The expression construct of any one of the preceding claims, wherein the NPC1 promoter fragment nucleotide sequence consists of SEQ ID NO: 1 and the hNPC1 nucleotide sequence consists of SEQ ID NO: 2 or 4.

10. A vector comprising the expression construct according to any one of claims 1 to 9.

11. The vector according to claim 10, which is a viral vector.

12. The vector according to claim 11, which is an adeno-associated virus (AAV) vector or comprises an AAV genome or a derivative thereof.

13. The vector according to claim 12, wherein said derivative is a chimeric, shuffled or capsid modified derivative.

14. The vector according to claim 12 or 13, wherein said AAV genome is from a naturally derived serotype or isolate or Glade of AAV.

15. The vector according to claim 14, wherein said AAV genome is from AAV serotype 2 (AAV2), AAV serotype 4 (AAV4), AAV serotype 5 (AAVS) or AAV serotype 8 (AAV8) and/or wherein the capsid is derived from AAV9.

16. The vector according to claim 15, wherein the genome is derived from AAV2 and the capsid is derived from AAV9.

17. A host cell that contains a vector of claim 10 or produces a viral vector of any one of claims 11 to 16.

18. The cell according to claim 17 that is a HEK293 or HEK293T cell.

19. A pharmaceutical composition comprising a vector of any one of claims 11 to 16 and a pharmaceutically acceptable carrier.

20. The vector according to any one of claims 11 to 16, or the pharmaceutical composition of claim 19, for use in medicine.

21. The vector according to any one of claims 11 to 16, or the pharmaceutical composition of claim 19, for use in a method of preventing or treating a disease associated with a loss of NPC1 function.

22. The vector or pharmaceutical composition for use according to claim 21, wherein the disease associated with a loss of NPC1 function is a lysosomal storage disorder.

23. The vector or pharmaceutical composition for use according to claim 22, wherein the lysosomal storage disorder is Niemann-Pick type C (NPC) disease.

24. Use of a vector according to any one of claims 11 to 16, or the pharmaceutical composition of claim 19, in the manufacture of a medicament for the treatment or prevention of NPC disease.

25. A method of treating or preventing NPC disease in a patient in need thereof, comprising administering a therapeutically effective amount of a vector according to any one of claims 11 to 16, or the pharmaceutical composition of claim 19, to said patient.

26. The vector or pharmaceutical composition for use according to claims 20 to 23, the use of claim 24, or the method according to claim 25, wherein the vector or pharmaceutical composition is administered parentally, preferably intravenously, or intracerebroventricularly, or by intracisternal magna administration, to a patient.
